(19) United States
(12) Patent Application Publication
(10) Pub. No.: US 2022/0119762 A1
(43) Pub. Date: Apr. 21, 2022

(54) CELLULAR MICRO-MASONRY SYSTEM

(71) Applicant: University of Florida Research Foundation, Inc., Gainesville, FL (US)

(72) Inventors: Sarah V. ELLISON, Gainesville, FL (US); Thomas Ettor ANGELINI, Gainesville, FL (US); Scott Arthur BANKS, Gainesville, FL (US); Duane MITCHELL, Gainesville, FL (US); Cameron MORLEY, Gainesville, FL (US); Catherine FLORES, Gainesville, FL (US)

(21) Appl. No.: 17/565,972

(22) Filed: Dec. 30, 2021

Related U.S. Application Data
(63) Continuation-in-part of application No. PCT/US2020/040497, filed on Jul. 1, 2020.
(60) Provisional application No. 62/869,303, filed on Jul. 1, 2019.

(51) Int. Cl. C12N 5/00; C12N 5/09; G01N 1/30

(52) U.S. Cl. CPC C12N 5/0062; C12N 5/0693; C12N 2527/00; C12N 2513/00; C12N 2533/90; G01N 1/30

(57) ABSTRACT

Described herein are systems and methods relating to cellular micro-masonry. Systems and methods as described herein allow a user to create three-dimensional (3D) structures of cells disposed in a 3D culture medium. Systems and methods as described herein provide for the manipulation and construction of cellular structures on a single, cell-by-cell, basis.

### CROSS-REFERENCE TO RELATED APPLICATION

This application is a continuation-in-part of International Patent Application entitled “CELLULAR MICRO-MASONRY SYSTEM”, having serial number PCT/US2020/040497, with an international filing date of Jul. 1, 2020, which claims priority to U.S. Provisional Application entitled “CELLULAR MICRO-MASONRY SYSTEM,” having Ser. No. 62/869,303, filed on Jul. 1, 2019, both of which are entirely incorporated herein by reference.

### BACKGROUND

The spatial structure and patterning of cells found in developing and mature tissues exhibit a level of detail so exquisite that they have the appearance of being built by hand, one cell at a time, as if carefully placed by a micro-scale mason. Within the 3D bioprinting field, there is a nearly ubiquitous sentiment that building sizable tissues in a cell-by-cell manner will not be practical for decades. The current paradigm is to build coarse structures composed of the right cell mixture and let “self-assembly” do the rest of the job to achieve a functional tissue. This view is practical; if the goal is to produce large structures containing 10⁵-10⁹ cells to make functioning tissues implantation or drug screening, then building structures cell-by-cell will not be effective. By contrast, fundamental unanswered questions about embryonic development, the evolution of multicellular processes, and signaling in the immune system, can all be investigated using structures made from far less than 10⁵ cells—anywhere between 2 and 10⁴ cells. However, no cellular micro-masonry system (CMMS) exists for creating the effectively perfect 3D structures required for such research. Accordingly, there is a need to address the aforementioned deficiencies and inadequacies.

### SUMMARY

Described herein are cellular micro-masonry systems. Cellular micro-masonry systems as described herein can comprise: a translation system; an imaging system; and a three-dimensional (3D) culture medium wherein the 3D cell culture medium comprises a plurality of hydrogel particles and a liquid cell culture medium, wherein the hydrogel particles are swelled with the liquid cell culture medium to form a granular gel.

In embodiments according to the present disclosure, cellular micro-masonry systems as described herein can further comprise a suction generating system, a pressure generating system, or both coupled to the translation system.

In embodiments according to the present disclosure, the translation system of cellular micro-masonry systems as described herein can further comprise a micro-capillary.

In embodiments according to the present disclosure, the translation system of cellular micro-masonry systems as described herein can be configured to provide one or more of three cartesian translational degrees of freedom (X, Y, Z), one radial degree of freedom (R), one azimuthal degree of freedom (ϕ), and one polar degree of freedom (θ).

In embodiments according to the present disclosure, imaging systems of cellular micro-masonry systems as described herein can further comprise a multi-photon microscopy system. In embodiments according to the present disclosure, imaging systems of cellular micro-masonry systems as described herein can comprise an inverted microscope.

In embodiments according to the present disclosure, the 3D culture medium of cellular micro-masonry systems as described herein can have a yield stress such that the cell growth medium undergoes a phase change from a first solid phase to a second liquid phase upon application of a shear stress greater than the yield stress. In certain embodiments, the yield stress is on the order of 10 Pa. In certain embodiments, the yield stress is less than 100 Pa. In embodiments according to the present disclosure, the 3D culture medium of cellular micro-masonry systems as described herein is a Herschel-Buckley material. In embodiments according to the present disclosure, the 3D culture medium of cellular micro-masonry systems as described herein have a short thixotropic time (on the order of a second to a few seconds).

In embodiments according to the present disclosure, the concentration of hydrogel particles can be between 0.05% to about 1.0% by weight.

In embodiments according to the present disclosure, the hydrogel particles can have a size between about 0.1 μm to about 100 μm when swollen with the liquid cell culture medium.

In embodiments according to the present disclosure, the plurality of cells can be disposed in a region of the 3D cell culture medium.

Described herein are methods of cellular micro-masonry. In embodiments according to the present disclosure, methods of cellular micro-masonry, can comprise: providing a cellular micro-masonry system as described herein; providing one or more cells in the three-dimensional (3D) culture media: approaching one of the one or more cells with the translation system; engaging the one cell with the translation system using suction; translating the one cell with the translation system according to one or more Cartesian translational degrees of freedom, one radial degree of freedom, one azithumal degree of freedom, or one polar degree of freedom; and releasing the cell in a desired location.

In embodiments according to the present disclosure, methods of cellular micro-masonry can further comprise manually correcting errors before or after the releasing.

In embodiments according to the present disclosure, methods of cellular micro-masonry can further comprise discarding cells that are not suitable.

In embodiments according to the present disclosure, the approaching, engaging, translating, and releasing can be monitored by the user using an imaging system.

In embodiments of methods according to the present disclosure, the imaging system comprises a multi-photon microscope. In embodiments of methods according to the present disclosure, the imaging system comprises an inverted microscope.

In embodiments of methods according to the present disclosure, the 3D culture medium can have a yield stress such that the cell growth medium undergoes a phase change from a first solid phase to a second liquid phase upon application of a shear stress greater than the yield stress.

In embodiments of methods according to the present disclosure, the yield stress can be on the order of 10 Pa. In embodiments of methods according to the present disclosure, the yield stress can be less than 100 Pa.

In embodiments of methods according to the present disclosure, the concentration of hydrogel particles can be between 0.05% to about 1.0% by weight.

In embodiments of methods according to the present disclosure, hydrogel particles can have a size between about 0.1 μm to about 100 μm when swollen with the liquid cell culture medium.

In embodiments, the one or more cells are one or more tumor cells. In embodiments, the one or more tumor cells are mammalian breast cancer cells.

In embodiments, methods further comprise proliferating the one or more cells in 2D culture before providing them to the 3D culture medium.

In embodiments, methods as described herein further comprise labeling the one or more cells with a live-cell dye. In embodiments of systems as described herein, the live-cell dye is a fluorescent dye. In embodiments, the 3D cell culture medium further comprises one or more extracellular matrix components. In embodiments of methods as described herein, the 3D cell culture medium further comprises one or more extracellular matrix components.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1E are graphics illustrating aspects of the present disclosure. Traditional masonry (FIG. 1A), in its simplest form, represents a building method for producing essentially perfect structures without advanced tools or unique skills. The power of masonry can be employed to create perfect structures from cells (FIG. 1B), using the same traditional principle: building one “brick” at a time. In cellular micro-masonry, the mason's hands are replaced by a micro-capillary attached to a translation system (FIG. 1C). The micro-capillary is also attached to a suction/pressure generator that enables gently picking up a single cell, translating it to a new location, and depositing it (FIG. 1C). Cellular micro-masonry would be impossible without the right support medium; cells take time to adhere to one another and even if a detailed structure could be assembled quickly, it would just ball up into a spherical shape over the course of several hours to days (FIG. 1D). A 3D culture medium made from jammed microgels swollen in ordinary liquid growth media will be used (described in detail herein) that allows “source” cells to be randomly dispersed in space and held in place. Source cells are retrieved by the micro-capillary, arranged into a precise 3D structure, and allowed to mature in the supporting growth environment (FIG. 1F)

FIGS. 2A-2B illustrate a system and workflow according to the present disclosure. FIG. 2A illustrates an embodiment of a system according to the present disclosure. In the embodiment of FIG. 2A, the cellular micro-masonry system (CMMS) combines MP microscopy, 6-axis manipulation, micropipette aspiration of cells, a 3D culture medium made from jammed microgels, image analysis, controls, robotics, path planning, optimization, and 3D graphical design. An embodiment of the micro-masonry build process is illustrated in FIG. 2B (“UF” made from cells.).

FIGS. 3A-3B are photographs illustrating aspects of the present disclosure, namely 3D printing of cells for tissue culture. Glioblastoma tumors (green) and rings of activated T cells (red) were 3D printed into microgel growth media and time-lapse imaging was performed. FIG. 3A shows t=0 min and FIG. 3B shows t=12 hours.

FIGS. 4A-4D illustrate aspects of the present disclosure. (FIG. 4A) microgels as described herein can be (a1) granular-scale (>1 μm diameter), cross-linked, hydrogel particles that form (a2) a jammed solid. (a3) At the macroscale, the jammed microgels can form a homogeneous continuum permeated with cell growth media that yields at low applied stress. This 3D culture medium enables (FIG. 4A) bioprinting cell assemblies or (FIG. 4B) isolated cell dispersal. (FIG. 4C) Microgels' large mesh-size makes this medium permeable to nutrients, waste, and molecular reagents. (FIG. 4D) in an embodiment, MCF10A cells can be assembled into multicellular structures by 3D printing into the microgel growth medium. A cross-hash network, a four-lobed lemniscate, and a single loop are displayed to scale, relative to a push-pin.

FIGS. 5A-5G are photographs showing an embodiment of a manual version of cellular micro-masonry as described herein using a patch-clamp micromanipulation system and bright-field microscopy. The operator was able to identify cells, pick them up, translate them at speeds between 10 and 1000 μm/s, and create a linear structure within a few minutes. FIGS. 5F and 5G show a “before” and “after”, respectively, of an embodiment of cellular micro-masonry as described herein. Steps utilized to build structures via micro-masonry are illustrated in FIGS. 5A-5E, which demonstrate approach (FIG. 5A), suction (FIG. 5B), translation in one axis (FIG. 5C), translation in a second axis (FIG. 5D), and release (i.e. placement, FIG. 5E).

FIGS. 6A-6B illustrate an embodiment of a physiological structure (acini) that can be created according to systems and methods as described herein.

FIGS. 7A-7C are cartoons illustrating embodiments of in vitro acinus models according to the prior art.

FIG. 8 illustrates a typical course of acini development.

FIG. 9 is a comparison of in vivo acini and in vitro acini grown according to a three-dimensional (3D) tissue culture model.

FIG. 10 is a cartoon that illustrates healthy vs. malignant tissue growth.

FIG. 11 is a cartoon that illustrates disadvantages and problems of current in vitro models of 3D acini culture.

FIG. 12 discloses aspects of 3D cell culture media (also referred to herein as jammed microgels or a “liquid-like solid”).

FIG. 13 is a graph of modulus vs. frequency for a small amplitude oscillatory frequency sweet showing the application of a low amplitude shear strain (1%) at various frequencies.

FIG. 14 is a graph of a small amplitude oscillatory frequency sweep of modulus vs. concentration showing a plot of modulus at 1 Hz vs. concentration.

FIG. 15 is a graph of a unidirectional shear sweep showing shear stress vs. shear rate and the application of shear-rate from high to low and plotting shear stress at various shear rates.

FIG. 16 is a plot of a unidirectional shear sweep showing yield stress vs concentration.

FIGS. 17A-17E are representative images from a video of 3D printing cells showing times 0 (FIG. 17A), 1 (FIG. 17B), 2 (FIG. 17C), 3 (FIG. 17D), and 4 (FIG. 17E) of MCF-10A cells 3D printed with a calcein red dye into a jammed microgel comprising 2.2% polymer and having a yield stress of 0.25 Pa.

FIGS. 18A-18F are representative images from a video of 3D printing cells and extracellular matrix (ECM) material showing times 0 (FIG. 18A), 1 (FIG. 18B), 2 (FIG. 18C), 3 (FIG. 18D), 4 (FIG. 18E), and 5 (FIG. 18F) of 3T3 cells 3D printed with 2 mg/mL collagen I, a CMFDA cell tracker green dye into a jammed microgel comprising 2.2% polymer and having a yield stress of 0.25 Pa.

FIGS. 19A-19B are plots relating to MCF-10A cell viability showing adjusted relative ATP production over 24 hours of cells in 5% methacrylic acid (MAA), 17% MAA, 17% carboxybetaine methacrylate (CBMA), and classic 2D culture measured with a CellTiter Glo® kit (Promega, US).

FIGS. 20A-20E are embodiments of 3D printed cellular structures according to micro-masonry systems and methods described herein. Madin Darby Canine Kidney (MDCK) cells labeled with 5-chloromethylfluorescein diacetate (CMFDA) and cell mask red dyes. Cellular structures were printed in a jammed microgel comprising 5% MAA swollen in Dubecco's modified eagle medium (DMEM) with 10% fetal bovine serum (FBS) and 1% penicillin streptomycin (pen-strep).

FIGS. 21A-21B illustrate an embodiment of the growth of functioning acini in jammed microgels according to the present disclosure (FIG. 21A). FIG. 21B is a plot of shear stress vs. shear rate for a Matrigel®-permeated jammed microgel according to the present disclosure.

FIGS. 22A-22D are confocal microscopy images of aspects of the present disclosure. MDCK cells labeled with CMFDA are shown at T=0 (FIG. 22A), 3 (FIG. 22B), 5 (FIG. 22C), and 6 (FIG. 22D) in 3% MAA polymer swollen with FBS and pen-strep and 1 mg/mL Matrigel®.

FIGS. 23A-23C show another view of FIG. 22D (FIG. 23A) and a 60× center slice of a cellular structure therein after 6 days of culture (FIG. 23B). FIG. 23C is a cellular structure at 60× magnification that was fixed and stained after 10 days culture. Nuclear and membrane structures can be seen in FIG. 23C.

FIG. 24 is a cartoon representing an embodiment of a system and method for cellular micro-masonry according to the present disclosure.

FIGS. 25A-25H are screenshots from a video showing a needle tip moving in microgel in relation to two cells in culture according to the present disclosure at T=0 (FIG. 25A), 1 (FIG. 25B), 2 (FIG. 25C), 3 (FIG. 25D), 4 (FIG. 25E), 5 (FIG. 25F), 6 (FIG. 25G), and 7 (FIG. 25H).

FIG. 26 is a flow chart of an embodiment of a method 100 according to the present disclosure.

FIG. 27 is a flow chart of an embodiment of a method 200 according to the present disclosure.

FIG. 28 is a flow chart of an embodiment of a method 300 according to the present disclosure.

FIG. 29A-29H: (FIG. 29A) With the micromasonry technique, such as the embodiment shown, cellular structures are precisely assembled in 3D space within a microgel-based culture medium that provides stabilizing support. Dispersed cells can be retrieved, translated, and deposited using a micromanipulator equipped with a glass microcapillary connected to a suction generator, all mounted on a confocal microscope. Shown in the present embodiment, a 3t3 fibroblast cells suspended in 3D is (FIG. 29B) approached and (FIG. 29C) retrieved by applying a light suction. (FIGS. 29D,E) The cell is translated to a chosen location in 3D space and (FIG. 29F) deposited. (FIG. 29G) Cells are dispersed in 3D; green cells are selected for assembly, while red cells are left in place (3t3 fibroblasts, false coloring). (FIG. 29H) The green cells are picked up, translated, and placed next to each other forming a single-file line of cells, suspended in 3D space.

FIG. 30A-30D: Two different MDCK cells populations are assembled into precise structures. (FIG. 30A) Red and green cells are dispersed randomly in the microgel medium. (FIG. 30B) Dispersed cells are retrieved and assembled into patterns with single cell precision, like the single file line with alternating cell colors, shown here. While hexagonal packing is expected for spheres, with micro-masonry such structures can be made with different patterning. (FIG. 30C) Square packings are possible and the emergence of collective behavior can be studied by increasing the dimensions of a given pattern. (FIG. 30D) Single cells can be arranged into large irregular shapes of specify design. See FIGS. 35A-35B for renderings of these objects when viewed from different angles in 3D.

FIG. 31A-31E Functional assays. (FIG. 31A) As a functional test a calcein transport assay was performed. Four red cells are assembled in a line and one cell dyed with calcein is placed on the end (top). All the cells become fluorescent green over 20 hours, indicating that gap junctions form allowing the calcein dye to travel from cell to cell. In control experiments, a cell dyed with CMFDA is placed on the end of the red-cell line; CMFDA is gap-junction impermeable (bottom). There appears to be no CMFDA transport in these experiments. (FIG. 31B) Space-time analysis of calcein fluorescence intensity shows the calcein transferring from the source cell to the neighboring cells. (FIGS. 31C, D, E) As a second functional assay, acini formation in the microgel medium is studied. (FIG. 31C) After five days hollow shells forming with disordered cytoskeletal structure can be seen. (FIGS. 31D,E) After 10 days, structures resembling mature acini can be seen (cyan: Hoescht; magenta: Alexa-phalloidin).

FIG. 32A-32E (FIG. 32A) Fluorescently labeled cells (GFP) from the sea anemone Nematostella vectensis embryos are disassociated, dispersed in the microgel medium, and used to build precise planar structures. (FIG. 32B) The disassociated embryonic cells are placed around a fibronectin-coated bead to observe their interactions with an anchoring surface in 3D (false colored; inset: uncolored bright-field image). (FIG. 32C) MDCK cells are dyed blue, red, and green to create three different populations. Three-color, 3D structures are assembled including a layered pyramid and (FIG. 32D) a layered stack of 2×2 structures. (FIG. 32E) A hollow spherical structure is built from MDCK cells dyed with CMFDA mimicking the structure of an acinus (left and center: volume-view renderings; right: X-Y slice).

FIG. 33A-33C: (FIG. 33A) A frequency sweep is performed from 10⁻² to 2 Hz. G′ is weakly dependent on frequency and always greater than G″ for both 5% and 6% MAA microgels. These rheological properties indicate that the microgel media is dominantly solid-like at low levels of strain. (FIG. 33B) To determine the yield stress of the microgel media, shear-rate sweeps are performed while measuring shear stress. The plateau in shear stress at low shear-rate corresponds to the yield stress of the microgel media, which are found to be between approximately 1.3 and 2.4 Pa. (FIG. 33C) While the viscosity of the microgel media is several orders of magnitude greater than water, cells experience extremely low shear stresses during micromasonry procedures because of the low shear-rates involved.

FIG. 34A-34B: MDCK cells are dyed with cell tracker green and dispersed in 5% MAA microgel media. (FIG. 34A) A glass microcapillary is translated rightward, passing two cells. The cells' initial positions are marked with a “+”. The cell closest to the capillary displaces by more than 1 cell diameter; the cell further from the capillary displaces by less than one diameter. (FIG. 34B) The needle tip is translated leftward, back past the cells. Both cells are moved toward their initial locations, having a final displacement of less than one cell diameter.

FIG. 34A-34C: In an embodiment, the microcapillary is translated right and left, past two MDCK cells, through 5% MAA microgel media. (FIG. 34A) False coloring the initial time-point in green and the final time-point in red, an overlay image shows the net displacements through a visible red and green patchiness. (FIG. 35B) PIV was performed to quantify the displacement field throughout the field of view. The measured velocity field is overlayed on a frame. (FIG. 35C) Creating a displacement map from the absolute values of the displacement vectors, it can be seen that displacements are patchy and largest near the microcapillary.

FIG. 35A-35B: MDCK cells are dyed with CMFDA or calcein red and dispersed in the jammed microgel medium. (FIG. 35A) The precise cellular structures are built one cell at a time as show in FIGS. 30A-30D in the main body. When viewing the 3D structures from different directions, using the volume viewer plugin in ImageJ, one can see the cells in a given assembly lay in a single chosen plane. (FIG. 35B) The same procedure demonstrates that the cells in the large-scale ‘UF’ structure are also co-planar.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of mechanical engineering, fluid motion, fluid dynamics, mechanical engineering, cellular biology, tissue culture, and the like.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

About: The term “about”, when used herein in reference to a value, refers to a value that is similar, in context to the referenced value. In general, those skilled in the art, familiar with the context, will appreciate the relevant degree of variance encompassed by “about” in that context.

Associated with: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition”, as used herein, can be used to refer to a discrete physical entity that comprises one or more specified components. In general, unless otherwise specified, a composition can be of any form—e.g., gas, gel, liquid, solid, etc.

Comprising: A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

“Jammed microgels”: As used herein, “jammed microgels” according to the present disclosure are hydrogel spheres packed tightly together enough that the material has a non-zero elastic shear-modulus.

“Improved,” “increased” or “reduced”: As used herein, these terms, or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: as used herein, a sample can be one or more cells or inorganic material whose position in 3D space is manipulated on a micrometer scale according to methods and systems described herein.

### Discussion

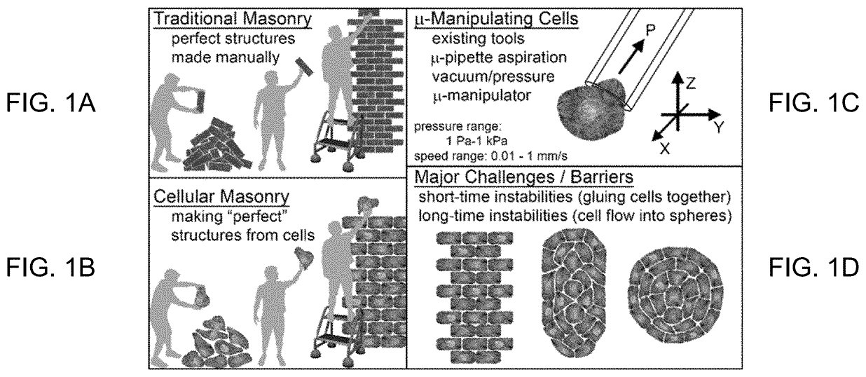

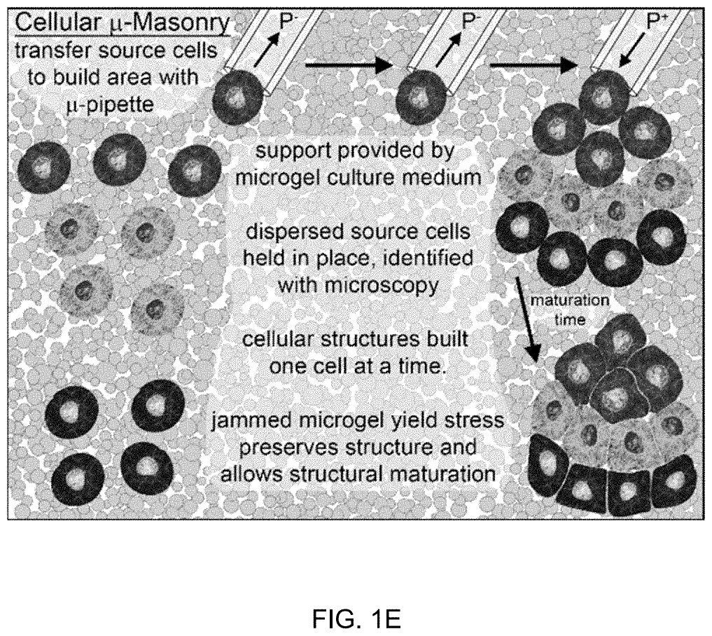

Described herein are systems and methods relating to cellular micro-masonry (or cellular masonry). As illustrated in FIGS. 1A-1E, Traditional masonry, in its simplest form, represents a building method for producing essentially perfect structures without advanced tools or unique skills. The power of masonry can be leveraged to create perfect structures from cells, using the same traditional principle: building one “brick” at a time. In cellular micro-masonry, the mason's hands are replaced by a micro-capillary attached to a translation system.

In embodiments, systems and methods as described herein comprise a translation system, an imaging system, and a suitable growth media. In embodiments, systems and methods as described herein can further comprise one or more living cells.

Translation systems as described herein can allow a user to change the position of a cell in a 3D culture medium from a first position in space to a second position in space, thereby translating the position of the cell in one or more axes. Translation systems can allow a user to move a cell along any one or more of the X-axis, Y-axis, or Z-axis of a 3D coordinate plane system. Translation systems as described herein can further allow for rotation of the cell in one or more axes. Translation systems as described herein can have repeatability of about 1/10 cell diameters, or approximately 1 μm, while traversing distances less than 1 mm.

In an embodiment, a translation system is a glass micropipette that can be manually aspirated and manipulated by a user.

In an addition embodiment, a translation system as described herein can be a disposable glass micro-capillary micropipette mechanically coupled to or fixed to 6-axis micromanipulation system with four translation and two rotation axes. Other micro-needles can also be suitable as long as it has an opening slightly smaller than the diameter of the cell which is translated (around 1 to 20 microns, for example). Translation stages can be comprised of a motorized manipulator, for example model MX7600L from Siskiyou. The translation stages can be moved by a user using a controller, such as the Siskiyou MC2010 controller, and instructions can be provided for translation of the translation stages through software such as LabVIEW by National Instruments. The translation stages can further be coupled to rotary stages for the user to rotate the cell among one or more axis. Rotary stages that can be coupled to the translation stages can be, for example, a motorized goniometer (for example, Physik Instrumente 65609211, controller model C-663.12), and/or a walking-piezo rotary stage (for example Physik Instrumente U-651.03, controller model C-867.1U). Other examples of translational systems according to the present disclosure can include other examples known in the art, for example the TransferMan® from Eppendorf.

The micro-capillary of the translation system can also be attached to a suction/pressure generator (for example a vacuum pump coupled to a pressure gauge, the micro-capillary connected by plastic tubing, for example) that enables gently picking up a single cell, translating it to a new location, and depositing it. The suction/pressure generator can be capable of generator/suction of about 1 Pa to 25 kPa.

In certain aspects, a pressure/vacuum generator and a micropipette puller can be utilized by systems and methods as described herein; 1 mm diameter glass microcapillaries can be connected to the pressure generator through polyethylene tubing and mounted onto the 6-axis assembly through the mounting system of embodiments of 4-axis micromanipulation systems as described herein, for examples those from Siskiyou.

Systems and methods relating to cellular micro-masonry further comprise an imaging system. An imaging system as described herein can be a multi-photon microscopy system (for example a Nikon A1R-MP), an epifluorescent microscopy system, a confocal microscopy system, a brightfield microscopy system, or other inverted imaging systems as known in the art.

Cellular micro-masonry would be impossible without the right support medium; cells take time to adhere to one another and even if a detailed structure could be assembled quickly, it would just ball up into a spherical shape over the course of several hours to days. In embodiments according to the present disclosure, systems and methods as described herein therefore use a 3D culture medium made from jammed microgels swollen in ordinary liquid growth media that allows “source” cells to be randomly dispersed in space and held in place. Source cells are retrieved by the micro-capillary, arranged into a precise 3D structure, and allowed to mature in the supporting growth environment. The 3D culture medium is described more in detail below.

3D Culture Medium

Liquid-like solid (LLS) three-dimensional (3D) cell growth medium (also referred to herein as “liquid-like solid”, “LLS”, “3D growth medium”, “3D cell growth medium”, “3D culture medium”; “granular microgel”; or “jammed microgel”) for use in with the disclosed bioreactor system is disclosed in WO2016182969A1 by Sawyer et al., which is incorporated by reference in its entirety for the description of how to make and uses this LLS medium.

Liquid-like solids (LLS) have properties that provide a combination of transport, elastic, and yielding properties, which can be leveraged to design a support material for the maintenance of living cells in three-dimensional culture. These materials may be composed predominantly of solvent that freely diffuses and can occupy more than 99% of their volume, but they also have a finite modulus and extremely low yield-stress in their solid state. Upon yielding, these materials shear and behave like classical fluids. Packed granular microgels are a class of liquid-like solids that have recently been adopted as a robust medium for precise three dimensional fabrication of delicate materials. The unrestricted diffusion of nutrients, small molecules, and proteins can support the metabolic needs of cells and provide an easy route to the development of combinatorial screening methods. Unperturbed, LLS materials can provide support and stability to cells and to cell-assemblies, and facilitate the development and maintenance of precise multi-cellular structures.

Briefly, the 3D cell growth medium may comprise hydrogel particles dispersed in a liquid cell growth medium. Any suitable liquid cell growth medium may be used; a particular liquid cell growth medium may be chosen depending on the types of cells which are to be placed within the 3D cell growth medium, as one of skill in the art would understand. For example, suitable cell growth medium may be human cell growth medium, murine cell growth medium, bovine cell growth medium or any other suitable cell growth medium. Depending on the particular embodiment, hydrogel particles and liquid cell growth medium may be combined in any suitable combination. For example, in some embodiments, a 3D cell growth medium comprises approximately 0.5% to 1% hydrogel particles by weight. In some embodiments, the hydrogel particles can have a size in the range of about 0.1 μm to about 100 μm when swollen with the liquid cell culture medium. In some embodiments, the hydrogel particles can have a size in the range of about 1 μm to about 10 μm when swollen with the liquid cell culture medium.

In accordance with some embodiments, the hydrogel particles may be made from a bio-compatible polymer.

The hydrogel particles may swell with the liquid growth medium to form a granular gel material. Depending on the particular embodiment, the swollen hydrogel particles may have a characteristic size at the micron or submicron scales. For example, in some embodiments, the swollen hydrogel particles may have a size between about 0.1 μm and 100 μm. Furthermore, a 3D cell growth medium may have any suitable combination of mechanical properties, and in some embodiments, the mechanical properties may be tuned via the relative concentration of hydrogel particles and liquid cell growth medium. For example, a higher concentration of hydrogel particles may result in a 3D growth medium having a higher elastic modulus and/or a higher yield stress.

According to some embodiments, the 3D cell growth medium may be made from materials such that the granular gel material undergoes a temporary phase change due to an applied stress (e.g. a thixotropic or “yield stress” material). Such materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Stress may be applied to such materials in any suitable way. For example, energy may be added to such materials to create a phase change. The energy may be in any suitable form, including mechanical, electrical, radiant, or photonic, etc.

Regardless of how cells are placed in the medium, the yield stress of the yield stress material may be large enough to prevent yielding due to gravitational and/or diffusional forces exerted by the cells such that the position of the cells within the 3D growth medium may remain substantially constant over time. As described in more detail below, placement and/or retrieval of groups of cells may be done manually or automatically.

A yield stress material as described herein may have any suitable mechanical properties. For example, in some embodiments, a yield stress material may have an elastic modulus between approximately 1 Pa and 1000 Pa when in a solid phase or other phase in which the material retains its shape under applied stresses at levels below the yield stress. In some embodiments, the yield stress required to transform a yield stress material to a fluid-like phase may be between approximately 1 Pa and 1000 Pa. In some embodiments, the yield stress may be on the order of 10 Pa, such as 10 Pa+/−25%. When transformed to a fluid-like phase, a yield stress material may have a viscosity between approximately 1 Pa s and 10,000 Pa s. However, it should be understood that other values for the elastic modulus, yield stress, and/or viscosity of a yield stress material are also possible, as the present disclosure is not so limited.

A group of cells may be placed in a 3D growth medium made from a yield stress material via any suitable method. For example, in some embodiments, cells may be injected or otherwise placed at a particular location within the 3D growth medium with a syringe, pipette, or other suitable placement or injection device, such as automated liquid handler. In some embodiments an array of automated cell dispensers may be used to inject multiple cell samples into a container of 3-D growth medium. Movement of the tip of a placement device through the 3D growth medium may impart a sufficient amount of energy into a region around the tip to cause yielding such that the placement tool may be easily moved to any location within the 3D growth medium. In some instances, a pressure applied by a placement tool to deposit a group of cells within the 3D growth medium may also be sufficient to cause yielding such that the 3D growth medium flows to accommodate the group of cells. Movement of a placement tool may be performed manually (e.g. “by hand”), or may performed by a machine or any other suitable mechanism.

In some embodiments, multiple independent groups of cells may be placed within a single volume of a 3D cell growth medium. For example, a volume of 3D cell growth medium may be large enough to accommodate at least 2, at least 5, at least 10, at least 20, at least 50, at least 100, at least 1000, or any other suitable number of independent groups of cells. Alternatively, a volume of 3D cell growth medium may only have one group of cells. Furthermore, it should be understood that a group of cells may comprise any suitable number of cells, and that the cells may of one or more different types.

Depending on the particular embodiment, groups of cells may be placed within a 3D cell growth medium according to any suitable shape, geometry, and/or pattern. For example, independent groups of cells may be deposited as spheroids, and the spheroids may be arranged on a 3D grid, or any other suitable 3D pattern. The independent spheroids may all comprise approximately the same number of cells and be approximately the same size, or alternatively different spheroids may have different numbers of cells and different sizes. In some embodiments, cells may be arranged in shapes such as embryoid or organoid bodies, tubes, cylinders, toroids, hierarchically branched vessel networks, high aspect ratio objects, thin closed shells, or other complex shapes which may correspond to geometries of tissues, vessels or other biological structures.

According to some embodiments, a 3D cell growth medium made from a yield stress material may enable 3D printing of cells to form a desired pattern in three dimensions. For example, a computer-controlled injector tip may trace out a spatial path within a 3D cell growth medium and inject cells at locations along the path to form a desired 3D pattern or shape. Movement of the injector tip through the 3D cell growth medium may impart sufficient mechanical energy to cause yielding in a region around the injector tip to allow the injector tip to easily move through the 3D cell growth medium, and also to accommodate injection of cells. After injection, the 3D cell growth medium may transform back into a solid-like phase to support the printed cells and maintain the printed geometry. However, it should be understood that 3D printing techniques are not required to use a 3D growth medium as described herein.

According to some embodiments, a 3D cell growth medium may be prepared by dispersing hydrogel particles in a liquid cell growth medium. The hydrogel particles may be mixed with the liquid cell growth medium using a centrifugal mixer, a shaker, or any other suitable mixing device. During mixing, the hydrogel particles may swell with the liquid cell growth medium to form a material which is substantially solid when an applied shear stress is below a yield stress, as discussed above. After mixing, entrained air or gas bubbles introduced during the mixing process may be removed via centrifugation, agitation, or any other suitable method to remove bubbles from 3D cell growth medium.

In some embodiments, preparation of a 3D cell growth medium may also involve buffering to adjust the pH of a hydrogel particle and liquid cell growth medium mixture to a desired value. For example, some hydrogel particles may be made from polymers having a predominantly negative charge which may cause a cell growth medium to be overly acidic (have a pH which is below a desired value). The pH of the cell growth medium may be adjusted by adding a strong base to neutralize the acid and raise the pH to reach the desired value. Alternatively, a mixture may have a pH that is higher than a desired value; the pH of such a mixture may be lowered by adding a strong acid. According to some embodiments, the desired pH value may be in the range of about 7.0 to 7.4, or, in some embodiments 7.2 to 7.6, or any other suitable pH value which may, or may not, correspond to in vivo conditions. The pH value, for example may be approximately 7.4. In some embodiments, the pH may be adjusted once the dissolved CO2 levels are adjusted to a desired value, such as approximately 5%.

Yield stress can be measured by performing a strain rate sweep in which the stress is measured at many constant strain rates. Yield stress can be determined by fitting these data to a classic Herschel-Bulkley model (σ=σ_{y}+k\dot{γ}ⁿ). (b) To determine the elastic and viscous moduli of non-yielded LLS media, frequency sweeps at 1% strain can be performed. The elastic and viscous moduli remain flat and separated over a wide range of frequency, behaving like a Kelvin-Voigt linear solid with damping. Together, these rheological properties demonstrate that a smooth transition between solid and liquid phases occurs with granular microgels, facilitating their use as a 3D support matrix for cell printing, culturing, and assaying.

An example of a hydrogel with which some embodiments may operate is a carbomer polymer, such as Carbopol®. Carbomer polymers may be polyelectrolytic, and may comprise deformable microgel particles. Carbomer polymers are particulate, high-molecular-weight crosslinked polymers of acrylic acid with molecular weights of up to 3-4 billion Daltons. Carbomer polymers may also comprise co-polymers of acrylic acid and other aqueous monomers and polymers such as poly-ethylene-glycol.

While acrylic acid is a common primary monomer used to form polyacrylic acid the term is not limited thereto but includes generally all α-β unsaturated monomers with carboxylic pendant groups or anhydrides of dicarboxylic acids and processing aids as described in U.S. Pat. No. 5,349,030. Other useful carboxyl containing polymers are described in U.S. Pat. No. 3,940, 351, directed to polymers of unsaturated carboxylic acid and at least one alkyl acrylic or methacrylic ester where the alkyl group contains 10 to 30 carbon atoms, and U.S. Pat. Nos. 5,034,486; 5,034,487; and 5,034,488; which are directed to maleic anhydride copolymers with vinyl ethers. Other types of such copolymers are described in U.S. Pat. No. 4,062,817 wherein the polymers described in U.S. Pat. No. 3,940,351 contain additionally another alkyl acrylic or methacrylic ester and the alkyl groups contain 1 to 8 carbon atoms. Carboxylic polymers and copolymers such as those of acrylic acid and methacrylic acid also may be cross-linked with polyfunctional materials as divinyl benzene, unsaturated diesters and the like, as is disclosed in U.S. Pat. Nos. 2,340,110; 2,340,111; and 2,533,635. The disclosures of all of these U.S. Patents are hereby incorporated herein by reference for their discussion of carboxylic polymers and copolymers that, when used in polyacrylic acids, form yield stress materials as otherwise disclosed herein. Specific types of cross-linked polyacrylic acids include carbomer homopolymer, carbomer copolymer and carbomer interpolymer monographs in the U.S. Pharmocopia 23 NR 18, and Carbomer and C10-30 alkylacrylate crosspolymer, acrylates crosspolymers as described in PCPC International Cosmetic Ingredient Dictionary & Handbook, 12th Edition (2008).

Carbomer polymer dispersions are acidic with a pH of approximately 3. When neutralized to a pH of 6-10, the particles swell dramatically. The addition of salts to swelled Carbomer can reduce the particle size and strongly influence their rheological properties. Swelled Carbomers are nearly refractive index matched to solvents like water and ethanol, making them optically clear. The original synthetic powdered Carbomer was trademarked as Carbopol® and commercialized in 1958 by BF Goodrich (now known as Lubrizol), though Carbomers are commercially available in a multitude of different formulations.

Hydrogels may include packed microgels—microscopic gel particles, ˜5 μm in diameter, made from crosslinked polymer. The yield stress of Carbopol® is controlled by water content. Carbopol® yield stress can be varied between about 1 Pa to about 1000 Pa. Thus, both materials can be tuned to span the stress levels that cells typically generate. As discussed above, while materials may have yield stresses in a range of 1-1000 Pa, in some embodiments it may be advantageous to use yield stress materials having yield stresses in a range of 1-100 Pa or 10-100 Pa. In addition, some such materials may have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

In one non-limiting example, a 3D cell growth medium comprises approximately 0.2% to about 0.7% by mass Carbopol® particles (Lubrizol). The Carbopol® particles are mixed with and swell with any suitable liquid cell growth medium, as described above, to form a 3D cell growth medium which comprises approximately 99.3% to about 99.8% by mass cell growth medium. After swelling, the particles have a characteristic size of about I μm to about 10 μm. The pH of the mixture is adjusted to a value of about 7.4 by adding a strong base, such as NaOH. The resulting 3D cell growth medium is a solid with a modulus of approximately 100-300 Pa, and a yield stress of approximately 20 Pa. When a stress is applied to this 3D cell growth medium which exceeds this yield stress, the cell growth medium transforms to a liquid-like phase with a viscosity of approximately 1 Pa s to about 1000 Pa s. As described above, the specific mechanical properties may be adjusted or tuned by varying the concentration of Carbopol®. For example, 3D cell growth media with higher concentrations of Carbopol® may be stiffer and/or have a larger yield stress.

In an embodiment, a LLS can be prepared with 0.9% (w/v) Carbopol® ETD 2020 polymer (Lubrizol Co.) was dispersed in cell growth media under sterile conditions. The pH of the medium is adjusted by adding NaOH until pH 7.4 is reached under the incubation condition of 37° C. and 5% 002, and the completely formulated material is homogenized in a high-speed centrifugal mixer. Carbopol® ETD 2020 swells maximally at this pH, making it suitable for cell culture applications. The gel medium was incubated at 37° C. and 5% 002.

The hydrogels for the LLS may be dispersed in solutions (e.g., solutions with cell growth medium) in various concentrations to form the LLS. One example of a concentration is below 2% by weight. Another concentration example is approximately 0.5% to 1% hydrogel particles by weight, and another is approximately 0.2% to about 0.7% by mass.

Hydrogels may include packed microgels—microscopic gel particles, ˜5 μL in diameter, made from crosslinked polymer. The yield stress of Carbopol® is controlled by water content. Carbopol® yield stress can be varied between roughly 1 and 1000 Pa. Thus, both materials can be tuned to span the stress levels that cells typically generate. As discussed above, while materials may have yield stresses in a range of 1-1000 Pa, in some embodiments it may be advantageous to use yield stress materials having yield stresses in a range of 1-100 Pa or 10-100 Pa. In addition, some such materials may have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

Those skilled in the art will appreciate that materials having a yield stress will have certain thixotropic properties, such as a thixotropic time and a thixotropic index. As used herein, a thixotropic time is a time for shear stress to plateau following removal of a source of shear. The inventors recognize that thixotropic time may be measured in different ways. As used herein, unless indicated otherwise, thixotropic time is determined by applying to a material, for several seconds, a stress equal to 10 times the yield stress of the material, followed by dropping the stress to 0.1 times the yield stress. The amount of time for the shear rate to plateau following dropping of the stress is the thixotropic time.

As used herein, a thixotropic index (for a yield stress material) is defined as the ratio of viscosity at a strain-rate of 2 s¹ to viscosity at a strain-rate of 20 s¹.

Yield stress materials with desirable yield stresses may also have desirable thixotropic properties, such as desirable thixotropic indexes or thixotropic times. For example, desirable yield stress materials (including hydrogel materials having a yield stress below 100 Pascals, some of which are described in detail below, such as Carbopol® materials) may have thixotropic times less than 2.5 seconds, less than 1.5 seconds, less than 1 second, or less than 0.5 seconds, and greater than 0.25 seconds or greater than 0.1 seconds. An exemplary Carbopol® solution may exhibit a yield stress below 100 Pascals (and below 25 Pascals in some embodiments), as well as low thixotropic times. The thixotropic times of the Carbopol® solutions having a yield stress below 100 Pascals may be less than 2.5 seconds, less than 1.5 seconds, less than 1 second, or less than 0.5 seconds, and greater than 0.25 seconds or greater than 0.1 seconds.

In some embodiments, for hydrogel yield stress materials with a yield stress below 100 Pascals (including those discussed in detail below, like Carbopol® solutions), the thixotropic index is less than 7, less than 6.5, or less than 5, and greater than 4, or greater than 2, or greater than 1.

Desirable yield stress materials, like hydrogels such as the Carbopol® solutions described herein, may thus have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

Because of the yield stress behavior of yield stress materials, materials deposited into a yield stress material (such as through 3D printing techniques described herein) may remain fixed in place in the yield stress material, without the yield stress material or the deposited material needing to be cured or otherwise treated to reverse a phase change (e.g., by heating to cross-link, following printing). Rather, the yield stress materials permit an indefinite working time on deposition of materials inside yield stress materials, including printing of cell clusters within yield stress materials.

In another non-limiting embodiment, a method for preparing a 3D cell growth medium is described. The method begins when hydrogel particles are mixed with a liquid cell culture medium. Mixing may be performed with a mechanical mixer, such as a centrifugal mixer, a shaker, or any other suitable mixing device to aid in dispersing the hydrogel particles in the liquid cell culture medium. During mixing, the hydrogel particles may swell with the liquid cell culture medium to form a granular gel, as discussed above. In some instances, the mixing act may result in the introduction of air bubbles or other gas bubbles which may become entrained in the gel. Such entrained gas bubbles are removed at via centrifugation, gentle agitation, or any other suitable technique. The pH of the mixture may then be adjusted; a base may be added to raise the pH, or alternatively an acid may be added to lower the pH, such until the pH of the mixture reaches a desired value. In some embodiments, the final pH value after adjustment is about 7.4.

In an embodiment, systems and methods related to cellular micro-masonry as described herein comprises: (1) a 6-axis micromanipulation system with four translation and two rotation axes, plus control software to manipulate the translation and rotation axes; (2) a vacuum/pressure generator for picking and placing cells using glass microcapillaries.

3D Culture Medium with ECM Components

In embodiments according to the present disclosure, 3D culture medium as described herein can further comprise one or more extracellular matrix (ECM) components. Such ECM components can comprise fibrins, elastins, fibronectins, collagens, laminins, and the like that are known in the art. In certain aspects, 3D culture medium as described herein can comprise Matrigel® (which is a gelatinous protein mixture secreted by Engelbreth-Holm-Swarm mouse sarcoma cells). In certain aspects, 3D culture medium as described herein is swollen with Matrigel®.

Liquid Medium

Liquid medium composition as known in the art, that can be employed in addition to the 3D culture medium as described above to “swell” the microgels, must be considered from two perspectives: basic nutrients (sugars, amino acids) and growth factors/cytokines. Co-culture of cells often allows reduction or elimination of serum from the medium due to production of regulatory macromolecules by the cells themselves. The ability to supply such macromolecular regulatory factors in a physiological way is a primary reason 3D perfused co-cultures are used. A serum-free medium supplemented with several growth factors suitable for long-term culture of primary differentiated hepatocytes has been tested and found to support co-culture of hepatocytes with endothelial cells. ES cells are routinely maintained in a totipotent state in the presence of leukemia inhibitory factor (LIF), which activates gp130 signaling pathways. Several medium formulations can support differentiation of ES cells, with different cytokine mixes producing distinct patterns of differentiation. Medium replacement rates can be determined by measuring rates of depletion of key sugars and amino acids as well as key growth factors/cytokines. If cell culture medium with sodium bicarbonate is used, the environmental control can be provided by e.g. placing the module with bioreactor/reservoir pairs into a CO2 incubator.

In embodiment, liquid medium according to the present disclosure as a constituent of the 3D cell culture medium is one suitable for cell growth and proliferation according to known methods in the art for a particular cell type or types. For example, for MDCK cells a suitable liquid medium can be DMEM with 10% FBS and 1% pen-strep

Cells

A variety of different cells can be applied to the 3D growth medium of the disclosed systems. In some embodiments, these are normal human cells or human tumor cells. The cells may be a homogeneous suspension or a mixture of cell types. The different cell types may be seeded onto and/or into the medium sequentially, together, or after an initial suspension is allowed to attach and proliferate (for example, endothelial cells, followed by liver cells). Cells can be obtained from cell culture or biopsy. Cells can be of one or more types, either differentiated cells, such as endothelial cells or parenchymal cells, including nerve cells, or undifferentiated cells, such as stem cells or embryonic cells. In one embodiment, the medium is seeded with a mixture of cells including endothelial cells, or with totipotent/pluripotent stem cells which can differentiate into cells including endothelial cells, which will form “blood vessels”, and at least one type of parenchymal cells, such as hepatocytes, pancreatic cells, or other organ cells.

Cells can be cultured initially and then used for screening of compounds for toxicity. Cells can also be used for screening of compounds having a desired effect. For example, endothelial cells can be used to screen compounds which inhibit angiogenesis. Tumor cells (such as breast cancer cells or acini precursors) can be used to screen compounds for anti-tumor activity. Cells expressing certain ligands or receptors can be used to screen for compounds binding to the ligands or activating the receptors. Stem cells can be seeded, alone or with other types of cells. Cells can be seeded initially, then a second set of cells introduced after the initial bioreactor tissue is established, for example, tumor cells that grow in the environment of liver tissue. The tumor cells can be studied for tumor cell behaviors or molecular events can be visualized during tumor cell growth. Cells can be modified prior to or subsequent to introduction into the apparatus. Cells can be primary tumor cells from patients for diagnostic and prognostic testing. The tumor cells can be assessed for sensitivity to an agent or gene therapy. Tumor cell sensitivity to an agent or gene therapy can be linked to liver metabolism of set agent or gene therapy. Cells can be stem or progenitor cells and the stem or progenitor cells be induced to differentiate by the mature tissue. Mature cells can be induced to replicate by manipulation of the flow rates or medium components in the system.

Applications

Without intending to be limiting, systems and methods as described herein have many different applications, such as assisting with the identification of markers of disease; assessing efficacy of anti-cancer therapeutics; testing gene therapy vectors; drug development; screening; studies of cells, especially stem cells; studies on biotransformation, clearance, metabolism, and activation of xenobiotics; studies on bioavailability and transport of chemical agents across epithelial layers; studies on bioavailability and transport of biological agents across epithelial layers; studies on transport of biological or chemical agents across the blood-brain barrier; studies on acute basal toxicity of chemical agents; studies on acute local or acute organ-specific toxicity of chemical agents; studies on chronic basal toxicity of chemical agents; studies on chronic local or chronic organ-specific toxicity of chemical agents; studies on teratinogenicity of chemical agents; studies on genotoxicity, carcinogenicity, and mutagenicity of chemical agents; detection of infectious biological agents and biological weapons; detection of harmful chemical agents and chemical weapons; studies on infectious diseases; studies on the efficacy of chemical agents to treat disease; studies on the efficacy of biological agents to treat disease; studies on the optimal dose range of agents to treat disease; prediction of the response of organs in vivo to biological agents; prediction of the pharmacokinetics of chemical or biological agents; prediction of the pharmacodynamics of chemical or biological agents; studies concerning the impact of genetic content on response to agents; filter or porous material below microscale tissue may be chosen or constructed so as bind denatured, single-stranded DNA; studies on gene transcription in response to chemical or biological agents; studies on protein expression in response to chemical or biological agents; studies on changes in metabolism in response to chemical or biological agents; prediction of agent impact through database systems and associated models; prediction of agent impact through expert systems; and prediction of agent impact through structure-based models.

Notably systems and methods as described herein can be utilized for the building and selection of biological samples, for example selecting and translating one cell at a time.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

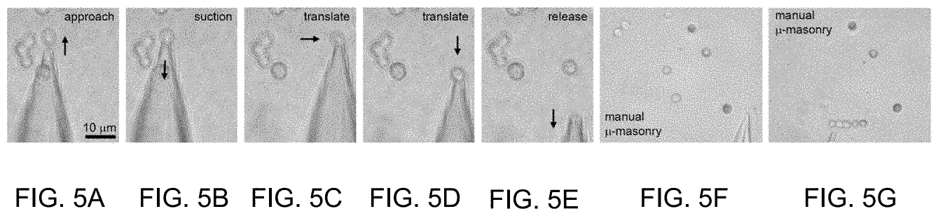

An embodiment of cellular micro-masonry was performed using a micro-manipulation system and bright-field microscopy. As shown in FIGS. 5A-5G, the operator identified cells, picked them up, translated them at speeds between 10 and 1000 μm/s, and created a simple linear structure within minutes.

Aspects of systems and methods as described herein are shown in FIGS. 5A-5E. The user can approach a cell in the 3D culture medium with the translation system (FIG. 5A), apply suction to engage with the cell (for example with a vacuum pump, FIG. 5B), translate the cell in one or more axes or coordinate planes (FIGS. 5C-5D), and release the cell at a desired position (FIG. 5E).

Micropipette aspiration is commonly used to apply suction to cells for measuring their elastic properties ^{28,29}. An embodiment of systems and methods as described herein demonstrating the ability to “pick-and-place” cells within the microgel-based culture medium (i.e. 3D culture medium) is shown in FIGS. 5A-5G.

A plurality of cells was manually dispersed, and then single cells were identified on an optical microscope. Using a translation system (for example the Siskiyou micromanipulation system as described herein) and imaging system, a cell in the 3D culture medium can be selected, the microcapillary tip can be moved to the cell's surface, a small amount of suction can be applied, the cell can be moved (i.e. translated), and placed by applying a small amount of positive pressure (FIG. 5G).

To provide the systems and methods with the capability of suction and/or pressure generation, a low-pressure testing system capable of generating suction and positive pressures covering a range of 1 Pa to 25 kPa can be used. This system can be operated by a “push button” panel or by interfacing with LabVIEW control software (Fluke 7250LP). Once this system is provided along with a glass micropipette puller (Sutter Instruments P-97) and integrated with the 6-axis manipulation system on the MP microscope, manual cellular micro-masonry can be realized.

Users can utilize systems and methods as described herein to build cellular structures manually, using hardware dials, joysticks, and buttons. Aspects of systems and methods can be tested by manually building structures from fluorescent microgel particles and living cells. Crude performance metrics can be assessed at this time, like ease-of-use, approximate pressure-levels required to suction and deposit cells, and acceptable translation speed ranges. To enable the systems and methods as described herein to be controlled by software, pressure/vacuum control for picking up a cell and placing it can be integrated into the LabVIEW GUI/control software as described herein. This step represents digital cellular micro-masonry as opposed to manual micro-masonry. Viability, expression of lineage specific markers by flow cytometry, and impact on cellular functions such as proliferation, migration, cytokine secretion, and in vitro killing function (cytotoxic T cell assays) with or without culture in the developed conditions can be investigated.

### Example 2

The need for improved tools and capabilities for experimentation with cells in 3D microenvironments and multi-cellular assemblies is widely recognized and remains a major challenge in cell biology and tissue engineering research. While the mechanical and materials aspects of the cellular micro-masonry system (CMMS) system and methods as described herein are designed to meet this major need, visualizing the cellular assemblies as they are built is critical to create designed structures with high fidelity and validate their quality. An imaging system can be employed, such as a fast-scanning multiphoton microscope (Nikon A1R-MP), to allow visualization by the user and to allow for visual feedback. According to this embodiment, with this microscope, single XY planes at full-field (approximately 1 mm×1 mm) can be collected at video rate (30 frames per second) while the user or control software scan through planes in the Z-direction to identify the location of a cell in 3D. Alternatively, “side-view” scans of the XZ or YZ planes can be collected at 10 frames per second. In certain aspects, the Nikon Jobs software package can be employed to design simple tools for quickly switching between the different perspectives, facilitating the pin-pointing of cell locations. The multi-photon functionality of this system is critical to this embodiments; with ordinary confocal microscopy, light cannot penetrate through multiple layers of cells, creating a shadowing effect. By contrast, multi-photon microscopy significantly reduces this problem enabling depths exceeding 1 mm to be imaged. Moreover, the long-wavelengths used in MP microscopy significantly reduce the effects of phototoxicity, enabling longer build-times with continuous illumination.

### Example 3

To provide flexibility to the user in creating 3D cell structures, in an embodiment, a micromanipulation system (i.e. translation system) can be employed with three cartesian translational degrees of freedom (X, Y, Z), one radial degree of freedom (R), one azimuthal degree of freedom (ϕ), and one polar degree of freedom (θ). The translation stages can be those, for example, from Siskiyou (model MX7600L) along with a programmable controller (Siskiyou MC2010) that can interface with LabVIEW (National Instruments, Austin, Tex.). GUI software, for example, written in LabVIEW, can be employed to allow a user to interface with and utilize the system. The 4-axis translation system can be mounted onto a motorized goniometer (for example Physik Instrumente 65609211, controller model C-663.12) that can mount onto the optical table next to the microscope base. To achieve rotation about the ϕ axis, the sample can be supported on a walking-piezo rotary stage mounted to the microscope stage (for example Physik Instrumente U-651.03, controller model C-867.1U). Other adapters, mounting systems, and supports can be designed and fabricated in machine shops by the skilled artisan to facilitate operation of the system. The system can be used by the user with manual controls (dials and joysticks), or in additional aspects, the system can be automated and interfaced with by a user through control software and a LabView GUI.

### Example 4

Cancer is the second highest cause of death for women. Breast cancer carries the highest cancer mortality rate, only behind lung cancer. 1 in 8 women will be diagnosed with breast cancer in her lifetime and 331,530 new cases of breast cancer are diagnosed in a year. There are 3.1 million cases of women that are being treated or that have been treated for breast cancer in the past year. Although there is an increased risk if a direct relative has had breast cancer, 85% of breast cancers occur in women with no family history, meaning genetic mutations occur in cells.

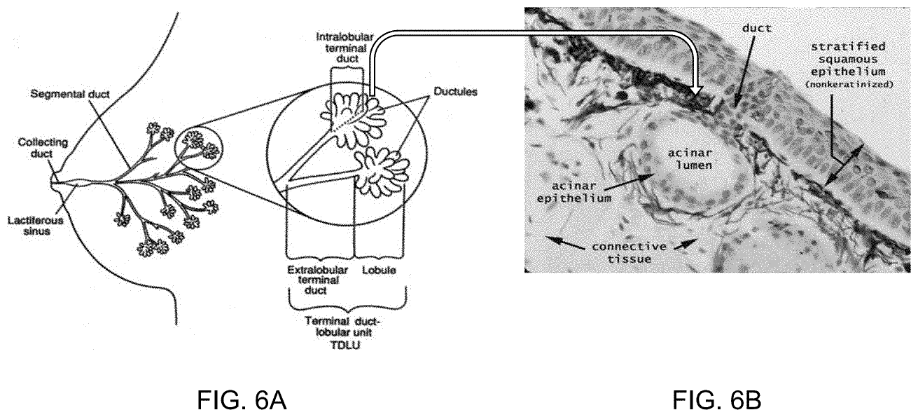

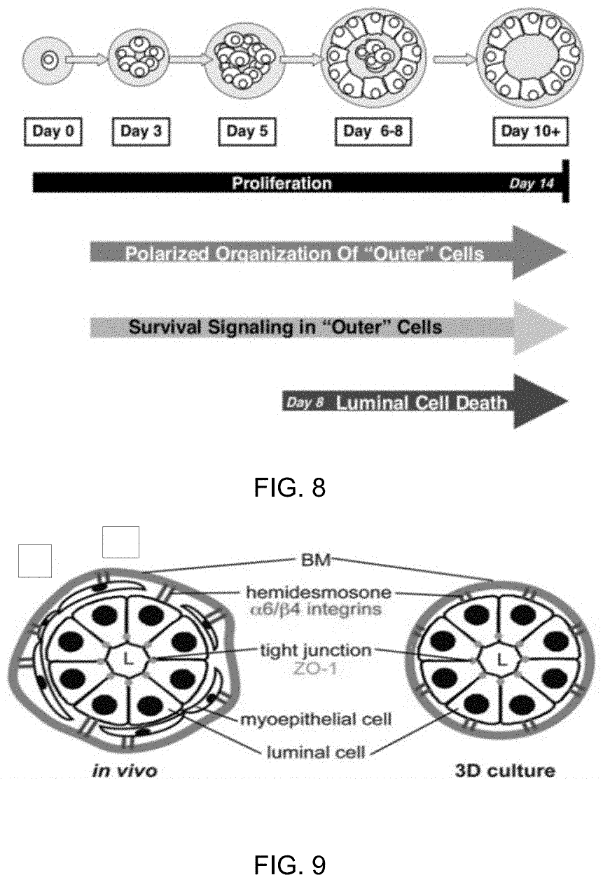

Breast cancer generally develops in acini. Acini are glandular breast tissues that constitute the “functional” breast tissue where milk is secreted (FIGS. 6A-6B). Acini continue to develop throughout lifetime, where they generally follow a greater-than-10-day developmental path that starts with proliferation, moves to the polarized organization of “outer” cells, survival signaling in “outer” cells, and luminal cell death after day 8 (FIG. 8). Acini are polarized, having an apical “free” or exposed surface and a basement membrane that regulates cell behavior.

Current cancel models generally rely on two-dimensional tissue culture and rodent studies. While necessary, Animal studies can be problematic. Not all cancers can grow in mice; cancers can take too long to grow; and it can be hard to observe tumor growth. It can also be desirable to reduce the numbers of animal subjects used in research.

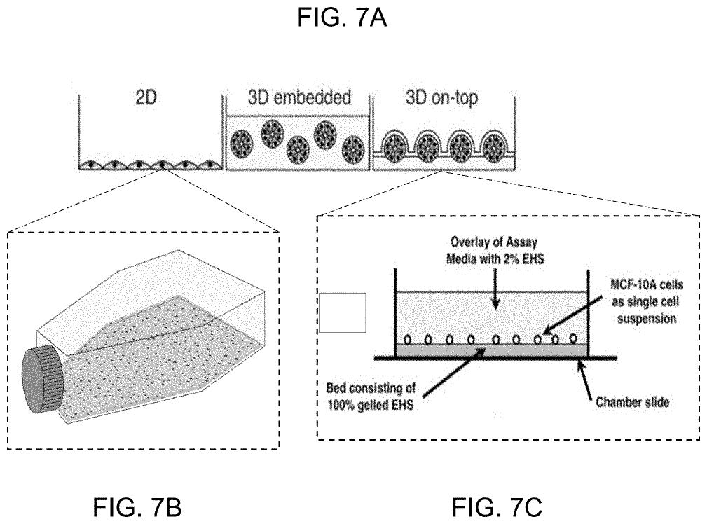

Tissue culture models for the study of breast cancer include 2D, three-dimensional (3D) embedded, and 3D on-top acinus models (FIGS. 7A-7C). MCF-10A cells are an immortalized line of mammary epithelial cells commonly used for study. Matrigel® (tumor-derived matrix consisting of laminin, collagen IV, and enactin) is another commonly utilized substrate for acini cultures.

2D studies also have issues, however, in that they don't recapitulate in vivo behavior; they cannot develop a polarized structure; and there is differential gene expression in 2D model structures than 3D models or xenografts. Additionally, stiff surfaces, such as polystyrene (3 GPa), stresses out cells. 3D culture models are more reliable as the structure of acini in 3D is more similar to in vivo growth in terms of at least basement membrane development; hemidesmosome development; tight junction development; and myoepithelial and luminal cell development (FIG. 9). The 3D structure of acini is shown to be highly correlated with their ability to function like in vivo tissue. A common method to observer in vitro structures includes removing from 3D growth media; fixing with a fixative (paraformaldehyde, for example) and staining for cellular and sub-cellular markers, such as E-cadherin to examine cell-cell tight junctions; GM130 for cell-basement membrane junctions; and laminin V and collagen IV antibodies to examine the basement membrane further.

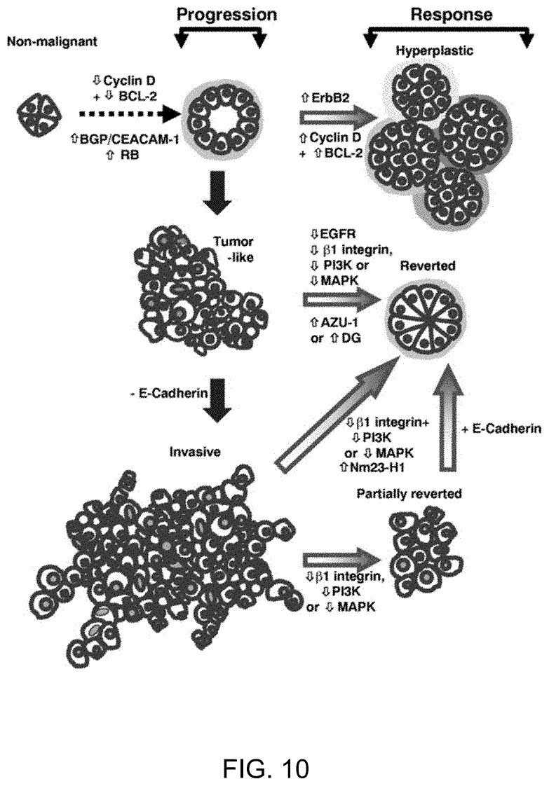

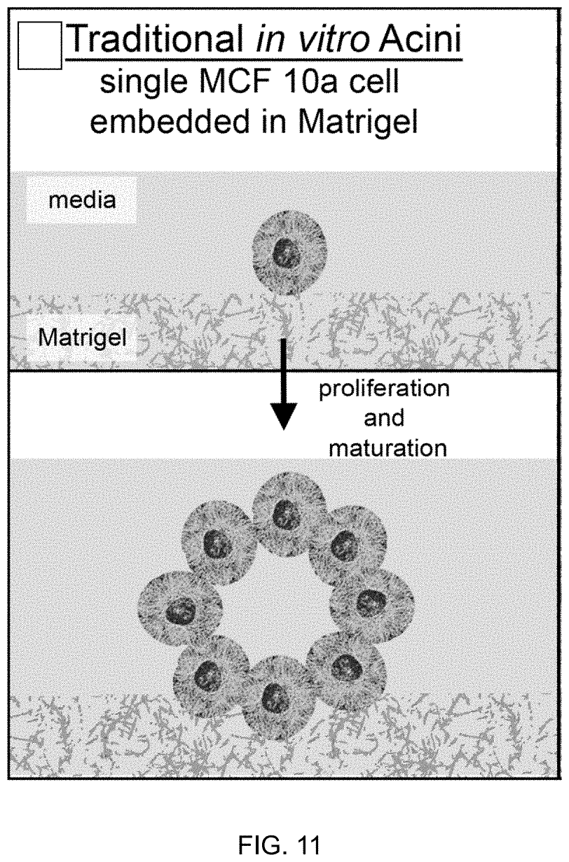

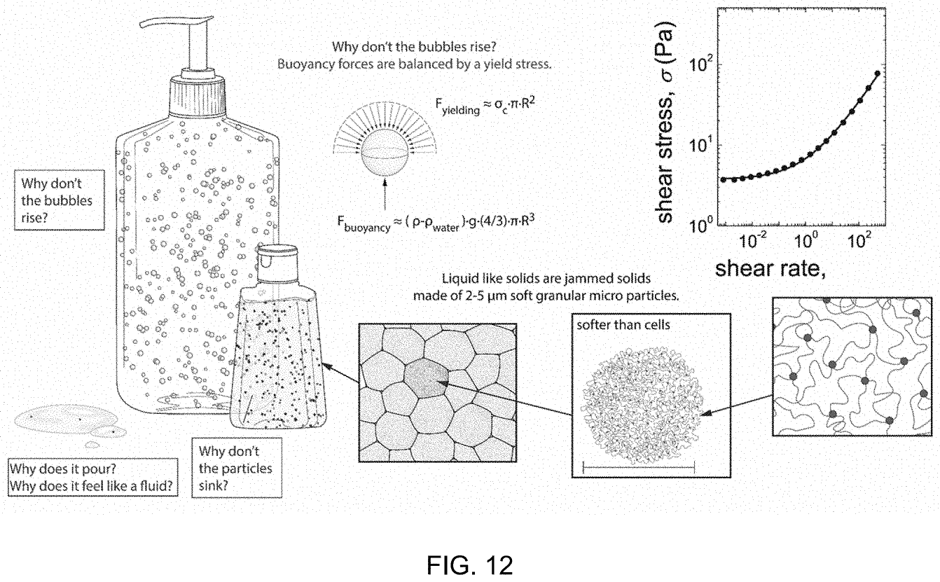

Healthy vs. malignant-like tissues can be observed in vitro (FIG. 10). Acini can be grown in 3D in a matrix such as Matrigel® (FIG. 11), and the structure of acini can be similar to in vivo. Malignant acini alter the culture medium (for example in terms of ECM compositions and/or depositions); appear disorganized (i.e. not spherical cells and forming heterogenous groups of cells); form heterogenous modules; and exhibit characteristics of carcinomas. The reversion of malignant-like to non-cancerous can also be observed in vitro as the shape can become more spherical over time and they can lack polarity. Lacking polarity can indicate non-functional acini, but spherical shape can indicate and non-cancerous and non-functional acini: aka (1) hollow spherical acini-functional (2) spherical but non-polarized: non-functional and non-cancerous (3) non-spherical, non-polarized, disorganized: cancerous

While 3D culture can be similar to in vivo regarding acini, additional issues persist. Cultures can grow slowly; they cannot interact after initial placement due to the mechanical properties of Matrigel®; the structure cannot be controlled; they can be difficult to image throughout development and across experimental studies. Accordingly, improvements to existing methods are presently desired.

As described herein, cellular micromasonry utilizing jammed microgels (also referred to herein as 3D cell culture medium or liquid-like solid) can be utilized to improve upon existing methods of 3D culture of acini (FIGS. 1A-1D). Jammed microgels according to the present disclosure are hydrogel spheres packed tightly together enough that the material has a non-zero elastic shear-modulus. Structures can be made cell-by-cell by micro-manipulating cells using existing tools, micropipette aspiration, vacuum/pressure, and a micro-manipulator. Operating pressure and speed ranges of the present methods and system can in the ranges of 1 pascal to 1 kilopascal with a speed range of 0.01 mm/s to 1 mm/s.

A micromanipulator can be used to move cells and build structures, but cells take time to adhere and structures can collapse without an appropriate growth/printing 3D medium to build cellular structures. Further described herein are jammed microgels (3D cell culture medium or liquid-like solids). Jammed microgels as described herein can be non-Newtonian fluids. In certain aspects, jammed microgels as described herein can be Herschel-Bulkley fluids. In certain aspects, jammed microgels as described herein can have a yield stress of less than 100 pascals. In certain aspects, jammed microgels as described herein comprise methacrylic acid carbomer polymers with a charge density of about 17 mol %. Jammed microgels can comprise one or more polymers swollen with a liquid medium, such as a cell culture medium. Such jammed microgels are softer than cells and can be microparticles in the size range of about 2 to 5 micrometers.

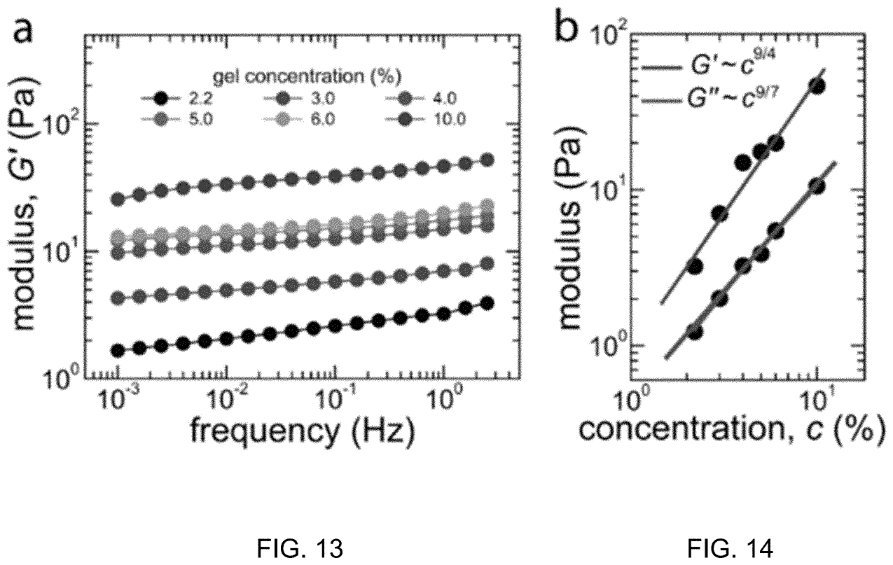

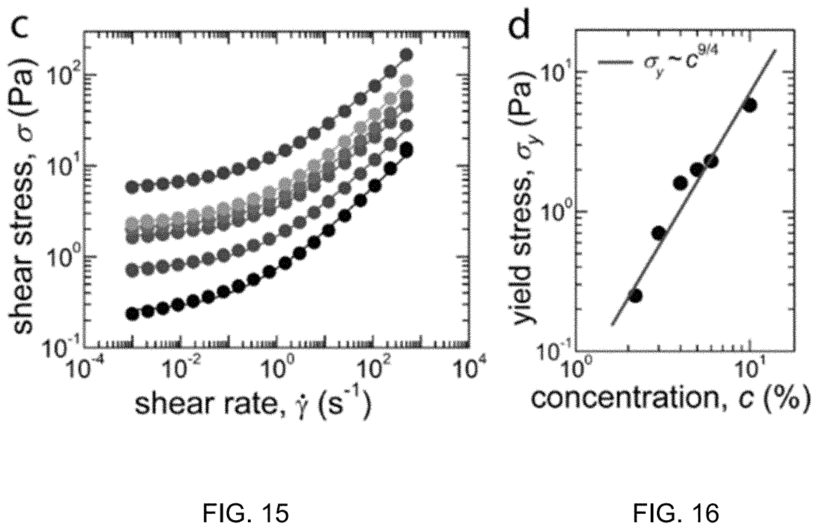

FIGS. 13-16 are plots illustrating aspects of microgel rheology for microgels as described herein. FIG. 13 is a graph of modulus vs. frequency for a small amplitude oscillatory frequency sweet showing the application of a low amplitude shear strain (1%) at various frequencies. FIG. 14 is a graph of a small amplitude oscillatory frequency sweep of modulus vs. concentration showing a plot of modulus at 1 Hz vs. concentration. FIG. 15 is a graph of a unidirectional shear sweep showing shear stress vs. shear rate and the application of shear-rate from high to low and plotting shear stress at various shear rates. FIG. 16 is a plot of a unidirectional shear sweep showing yield stress vs concentration.

FIGS. 13 and 14 represent observations of the time-dependent behavior of a material. The material can be placed between plates and it can be determined the force necessary to deform the material. Materials with a weak frequency dependence indicate an elastic solid (weak enough that slope is ignored). G′: storage modulus, elastic like behavior; G″: loss modulus, viscous like behavior; G′>G″: solid-like behavior; G′ rises with microgel concentration and exhibits a weak frequency dependence. clear dependence on concentration (G′˜c^{9/4}) can be treated as traditional polymers. Scales at 9/4 power law: characteristic of hydrogels due to mesh size and thermal fluctuations, near jamming follow traditional polymer physics.

FIGS. 15 and 16 are plots of a unidirectional shear sweep relating to observations on the transition between solid-like to fluid-like behavior. Unidirectional shear rheology: transition from solid-like behavior to fluid-like behavior with increasing shear rate. As the measured shear stress approaches a plateau as the shear rate decreases, which corresponds to the yield stress, σy. Materials as described herein can be fit to a Hershel-Bulkley model:

$$σ = {σ}_{y} ( 1 + {( \frac{\dot{γ}}{{\dot{γ}}_{c}} )}^{P} ) \qquad ( eq . 1 )$$

σ: applied stress; σ_{y}: yield stress; \dot{γ}: shear rate; \dot{γ}_{c}: crossover shear-rate between solid and liquid-like behaviors; and p: dimensionless order ˜0.5.

In determining yield stress, high shear-rate: stress varies; low shear-rate: independent of shear stress; determine crossover from solid-like to liquid-like behavior. There is a clear dependence on concentration clear dependence on concentration and σ_{y}˜c^{9/4} can be treated as traditional polymers.

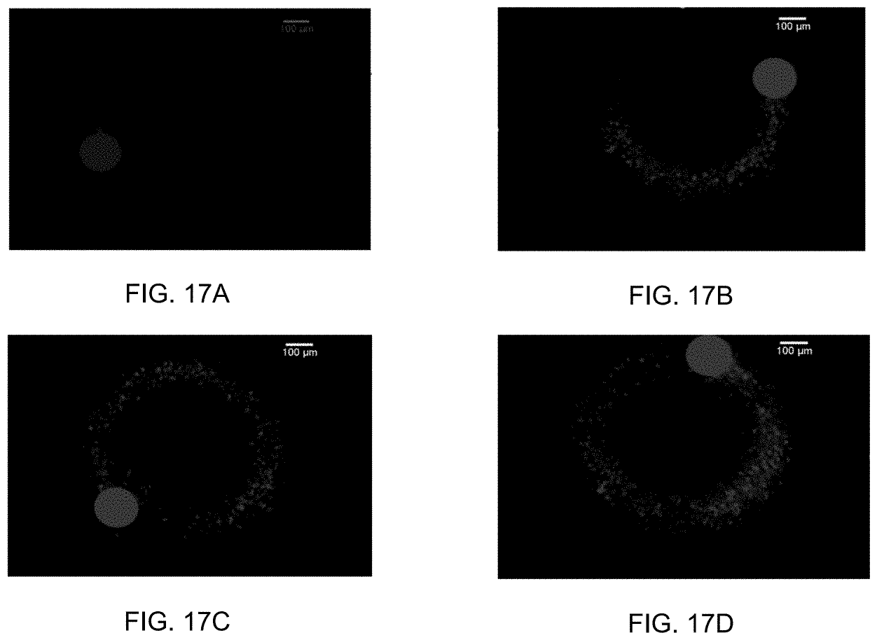

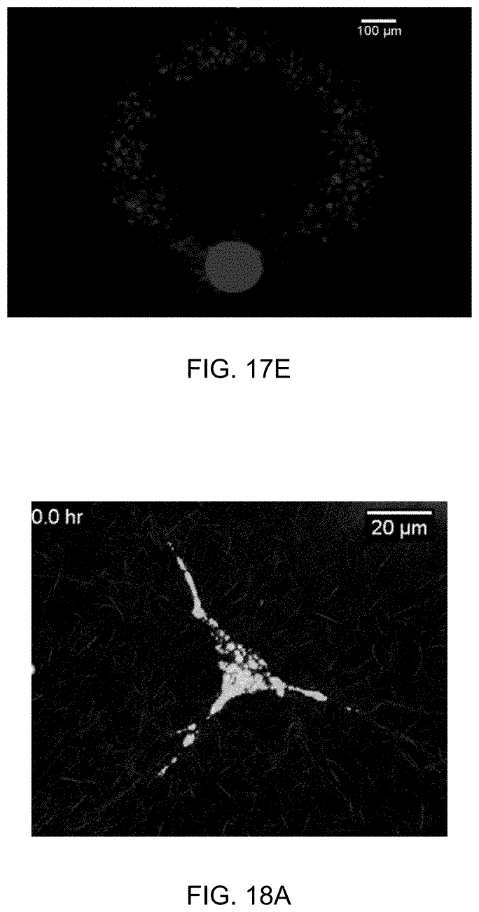

In certain aspects, cells utilized for 3D culture can be printed with a printing apparatus. Printing imparts greater control of cell placement to create 3D structures that is otherwise impossible by hand, but methods to date use lots of cells to print a structure and do not allow for single cell precision of printed structures. Examples of 3D printing are shown in FIGS. 17A-17E, 18A-18F, and 19A-19B.

FIGS. 17A-17E are representative images from a video of 3D printing cells showing times 0 (FIG. 17A), 1 (FIG. 17B), 2 (FIG. 17C), 3 (FIG. 17D), and 4 (FIG. 17E) of MCF-10A cells 3D printed with a calcein red dye into a jammed microgel comprising 2.2% polymer and having a yield stress of 0.25 Pa.

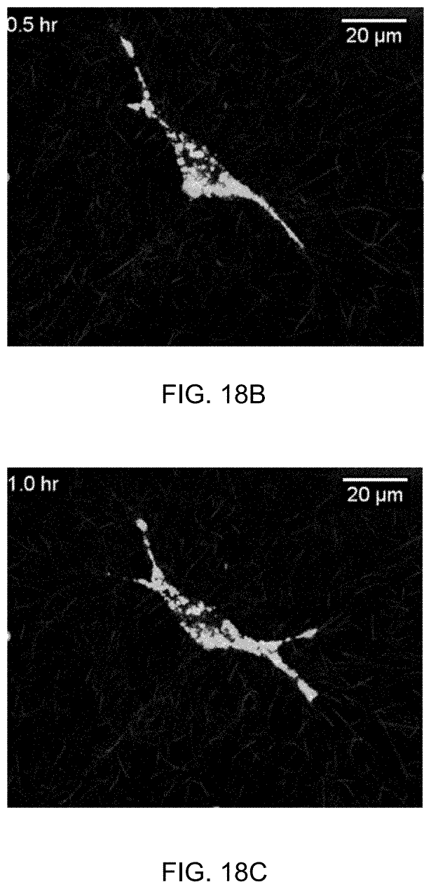

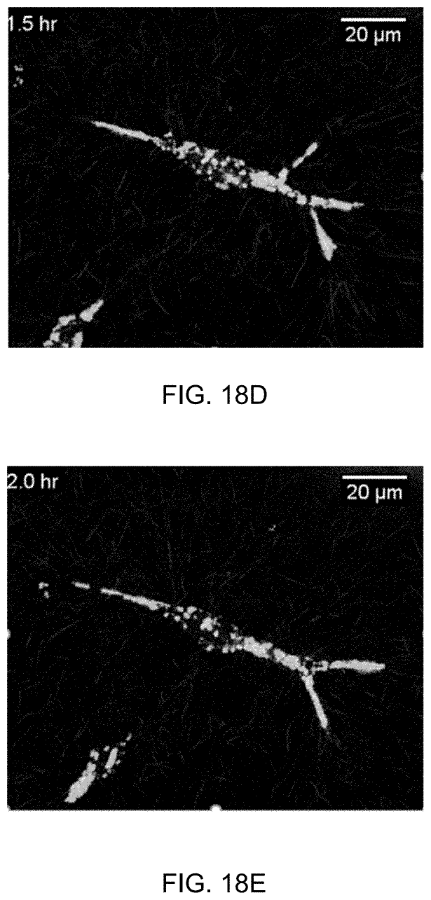

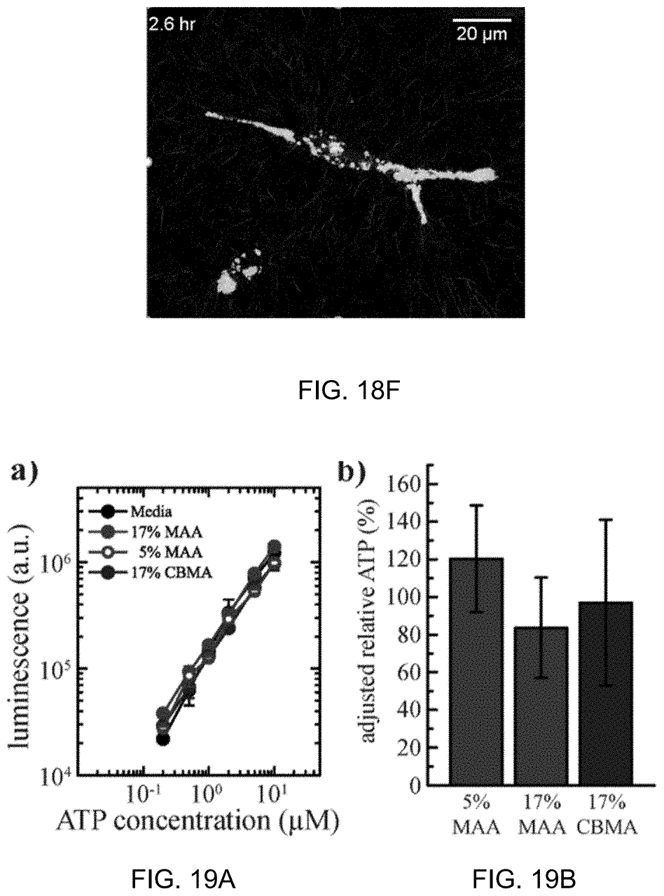

FIGS. 18A-18F are representative images from a video of 3D printing cells and extracellular matrix (ECM) material showing times 0 (FIG. 18A), 1 (FIG. 18B), 2 (FIG. 18C), 3 (FIG. 18D), 4 (FIG. 18E), and 5 (FIG. 18F) of 3T3 cells 3D printed with 2 mg/mL collagen I, a CMFDA cell tracker green dye into a jammed microgel comprising 2.2% polymer and having a yield stress of 0.25 Pa.

FIGS. 19A-19B are plots relating to cell viability showing adjusted relative ATP production over 24 hours of cells in 5% methacrylic acid (MAA), 17% MAA, and 17% carboxybetaine methacrylate (CBMA) measured with a CellTiter Glo® kit (Promega, US). Compared to 2D culture, ATP production over 24 hours is about the same as with 3D culture.

Described herein is a cellular micromasonry approach whereby single cells can be manipulated involving a 3-axis micromanipulator: x, y, z, tilt; external pressure source; and inverted microscope. For example, FIGS. 5A-5G are photographs showing an embodiment of a manual version of cellular micro-masonry as described herein using a patch-clamp micromanipulation system and bright-field microscopy. The operator was able to identify cells, pick them up, translate them at speeds between 10 and 1000 μm/s, and create a linear structure within a few minutes. FIGS. 5F and 5G show a “before” and “after”, respectively, of an embodiment of cellular micro-masonry as described herein. Steps utilized to build structures via micro-masonry are illustrated in FIGS. 5A-5E, which demonstrate approach (FIG. 5A), suction (FIG. 5B), translation in one axis (FIG. 5C), translation in a second axis (FIG. 5D), and release (i.e. placement, FIG. 5E). Moving forward, confocal microscopy can be combined with fluorescent imaging and methods and systems as described herein to build 3D structures, such as acini.

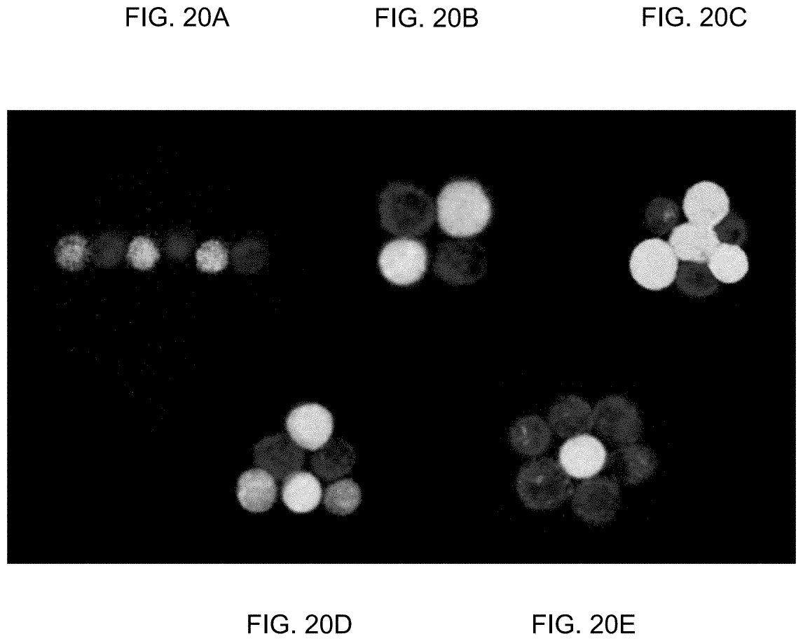

Such concepts are illustrated in the reduced-to-practice embodiments of FIGS. 20A-20E. FIGS. 20A-20E are embodiments of 3D printed cellular structures according to micro-masonry systems and methods described herein. Madin Darby Canine Kidney (MDCK) cells labeled with 5-chloromethylfluorescein diacetate (CMFDA) and cell mask red dyes. Cellular structures were printed in a jammed microgel comprising 5% MAA swollen in Dubecco's modified eagle medium (DMEM) with fetal bovine serum (FBS) and pen-strep.

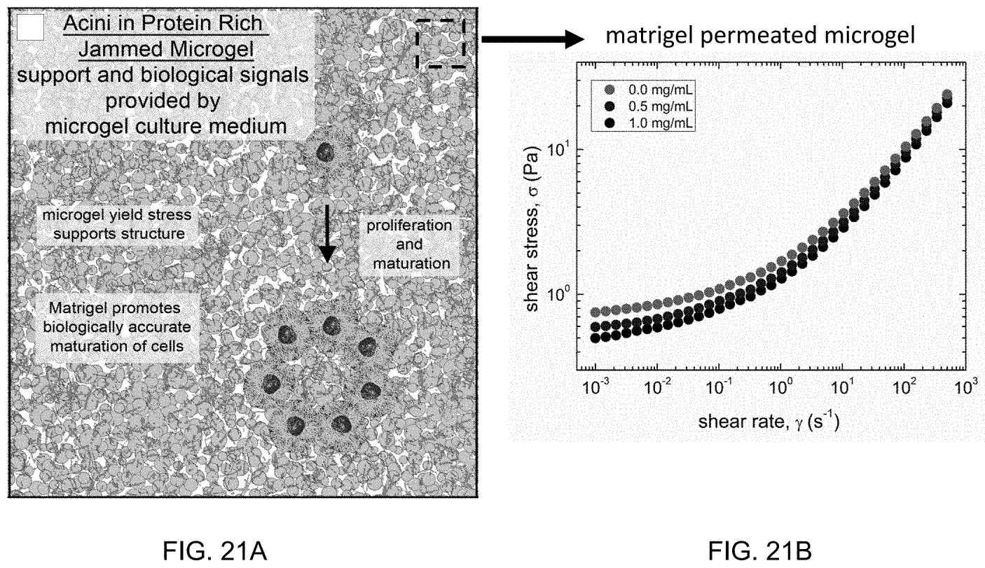

FIGS. 21A-21B illustrate an embodiment of the growth of functioning acini in jammed microgels according to the present disclosure (FIG. 21A). FIG. 21B is a plot of shear stress vs. shear rate for a Matrigel®-permeated jammed microgel according to the present disclosure.

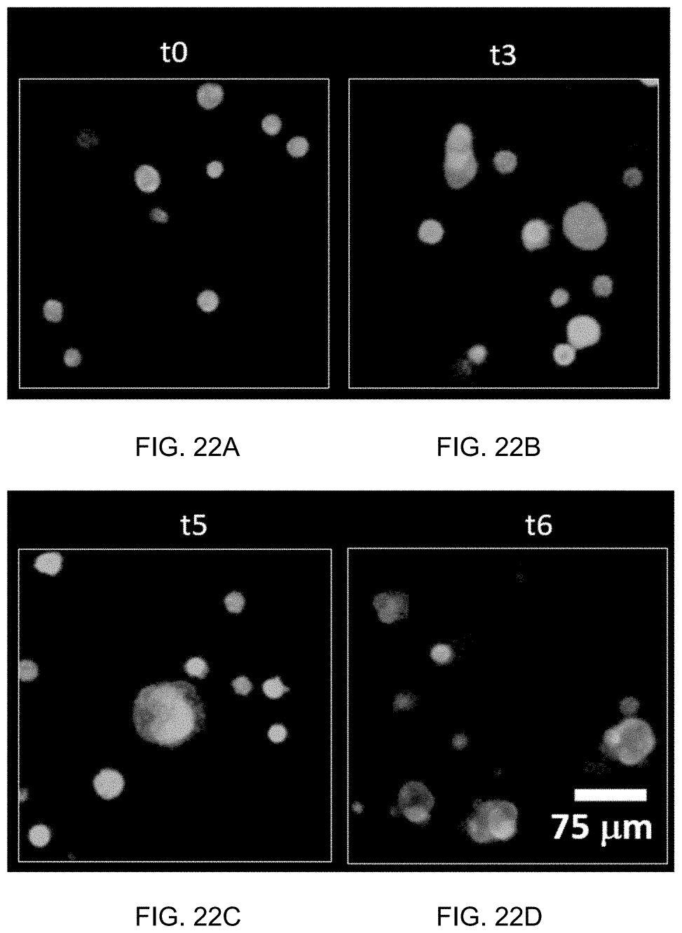

FIGS. 22A-22D are confocal microscopy images of aspects of the present disclosure. MDCK cells labeled with CMFDA are shown at T=0 (FIG. 22A), 3 (FIG. 22B), 5 (FIG. 22C), and 6 (FIG. 22D) in 3% MAA polymer swollen with FBS and pen-strep and 1 mg/mL Matrigel®.

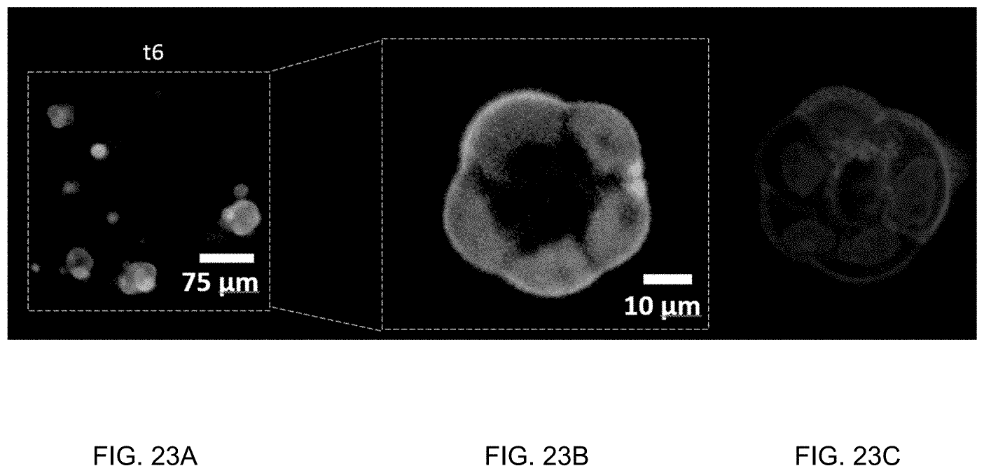

FIGS. 23A-23C show another view of FIG. 22D (FIG. 23A) and a 60× center slice of a cellular structure therein after 6 days of culture (FIG. 23B). FIG. 23C is a cellular structure at 60× magnification that was fixed and stained after 10 days culture. Nuclear and membrane structures can be seen in FIG. 23C.

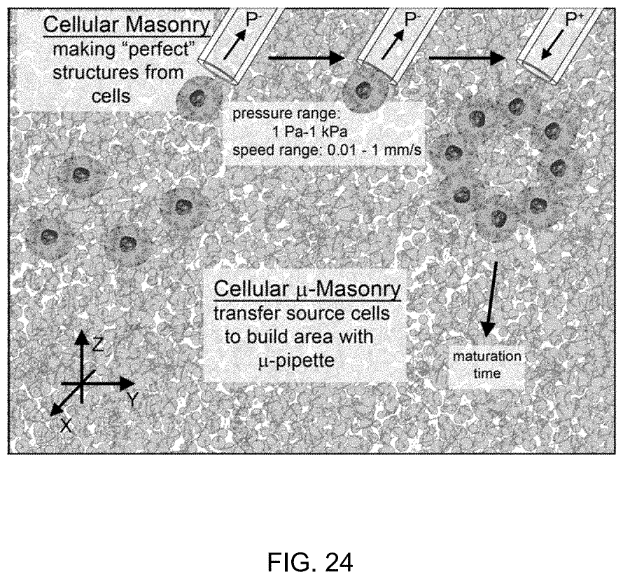

FIG. 24 is a cartoon representing an embodiment of a system and method for cellular micro-masonry according to the present disclosure, in particular relating to building an acinus structure. By first building the physical structure of an acinus cell-by-cell, in the biologically necessary growth factors, it is thought that the tissue may become physiologically functional more quickly than in current in vitro models, thereby improving upon existing models at least in this regard. Improved speed in becoming functional greatly reduces resources necessary for such experiments, including manpower and consumables, and improves the pace at which data can be generated and hypothesis validated and/or improved.

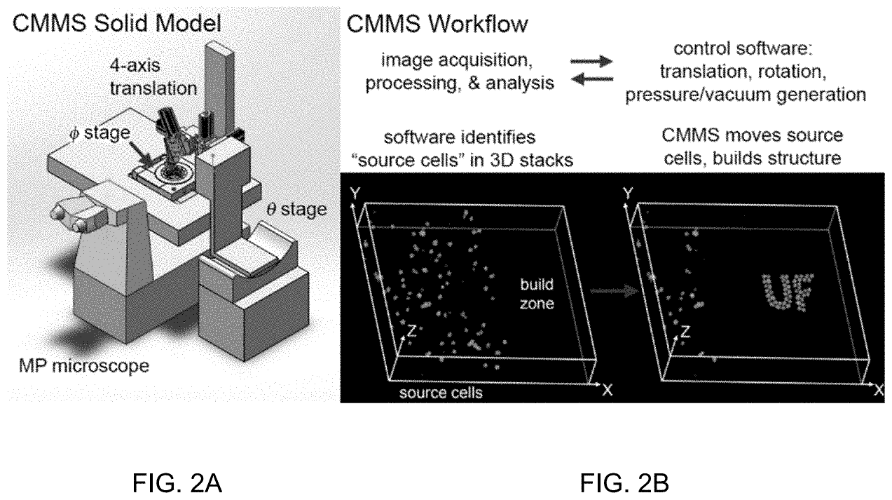

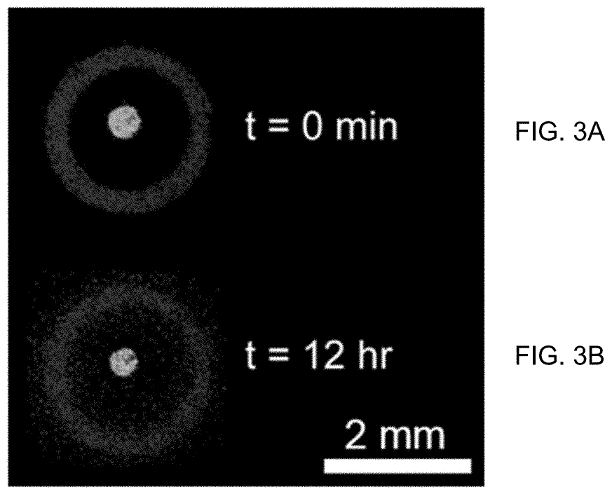

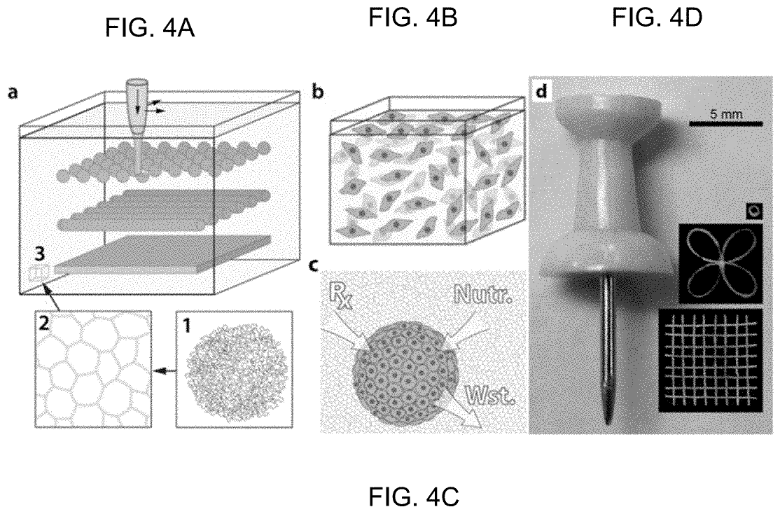

Briefly, according to systems and methods as described herein utilizing cellular micromasonry, it can be possible to print hollow spheres (approximately 2 mm in diameter) in Matrigel permeated microgel. Cells can first be grown and proliferated/expanded in 2D; dyed with a live-cell cellular tracker (such as CMFDA, for example); dispersed within the microgel using a translational apparatus as described herein; spheres can be constructed/built; visual assessment of the spheres can be undertaken; followed by other techniques such as immunostaining or gene expression analysis to study printed spheres. This process can be automated, for example as shown in FIGS. 2A-2B.

In an embodiment of 2D culture according to the present disclosure, cells can be maintained in standard polystyrene dishes, plates, or flasks. Liquid media can be exchanged every two days. When cell density reaches 70% confluence, the cells are detached from the plate with Trypsin, diluted in new liquid media, and seeded onto a new culture surfaces at 1/10 the density, starting the cycle over again.

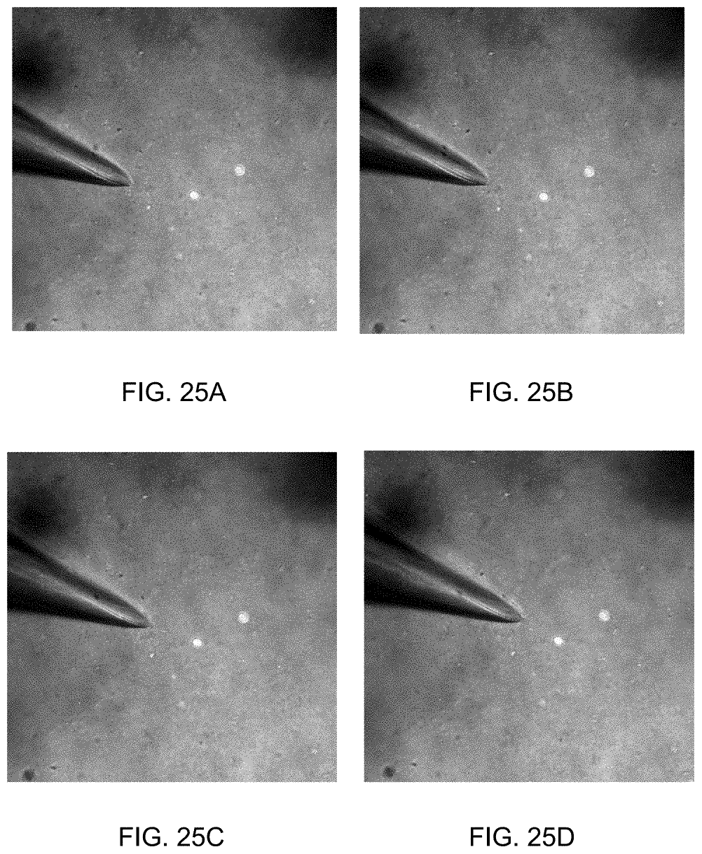

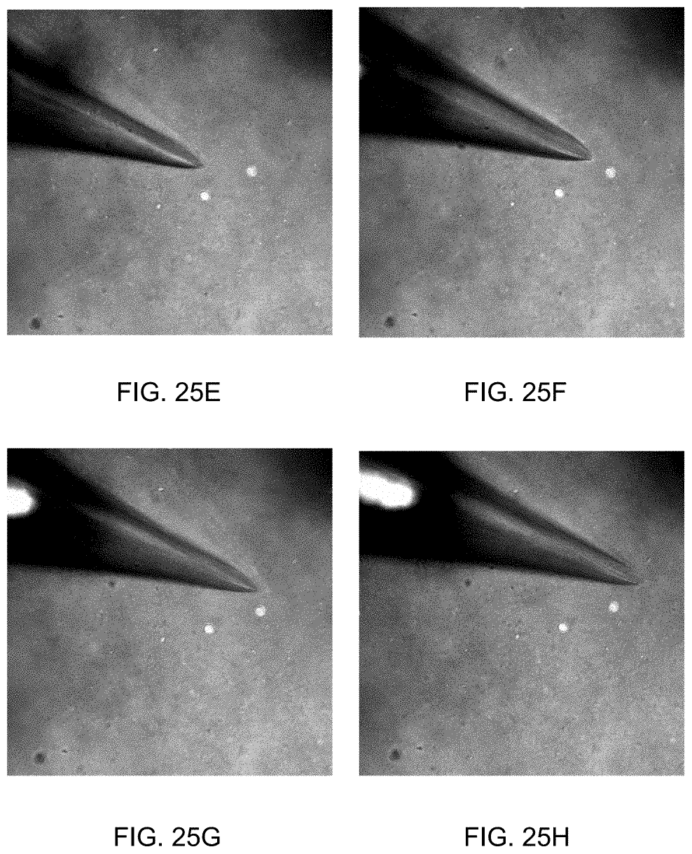

Vacuum suction and/or pressure can be utilized with translational apparati disclosed herein to deal with issues such as cells moving away from a deposited needle because of fluid motion, a problem exhibited in FIGS. 25A-25H. FIGS. 25A-25H are screenshots from a video showing a needle tip moving in microgel in relation to two cells in culture according to the present disclosure at T=0 (FIG. 25A), 1 (FIG. 25B), 2 (FIG. 25C), 3 (FIG. 25D), 4 (FIG. 25E), 5 (FIG. 25F), 6 (FIG. 25G), and 7 (FIG. 25H). Other aspects of the needle tip of the translational apparatus can be improved upon, such as making it fluorescent using means such as fluorescent pluronic; bovine serium albumin (BSA) rhodamine; N-(trimethoxysilylpropyl) ethylenediamine triacetic acid (TMS-EDTA); carboxylated silane; or Schott glass.

### Example 5

Disclosed herein are embodiments of methods of cellular micro-masonry.

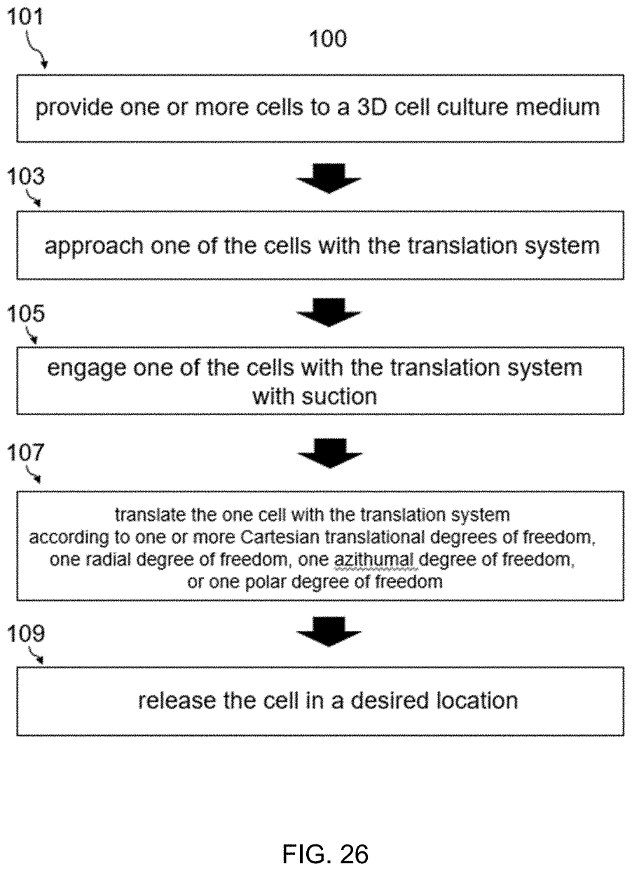

FIG. 26 is a flow chart of an embodiment of a method 100 according to the present disclosure.

In embodiments as described herein, a method 100 of cellular micro-masonry, comprises: providing one or more cells in the three-dimensional (3D) culture media 101: approaching one of the one or more cells with the translation system 103; engaging the one cell with the translation system using suction 105; translating the one cell with the translation system according to one or more Cartesian translational degrees of freedom, one radial degree of freedom, one azithumal degree of freedom, or one polar degree of freedom 107 (individually or in combination); and releasing the cell in a desired location 109.

In embodiments, methods can further comprise manually correcting errors before or after the releasing. In embodiments, methods can further comprise discarding cells that are not suitable.

In embodiments, the approaching, engaging, translating, and releasing are monitored by the user using an imaging system.

In embodiments, the imaging system is a multi-photon microscope. In embodiments, the 3D culture medium has a yield stress such that the cell growth medium undergoes a phase change from a first solid phase to a second liquid phase upon application of a shear stress greater than the yield stress.

In embodiments, the yield stress is on the order of 10 Pa. In embodiments, the concentration of hydrogel particles is between 0.05% to about 1.0% by weight.

In embodiments, the hydrogel particles have a size between about 0.1 μm to about 100 μm when swollen with the liquid cell culture medium.

In embodiments, the one or more cells are one or more tumor cells. In embodiments, the one or more tumor cells are mammalian breast cancer cells. In embodiments, methods as described herein further comprise proliferating the one or more cells in 2D culture before providing them to the 3D culture medium.

In embodiments, methods as described herein further comprise labeling the one or more cells with a live-cell dye. In embodiments, the live-cell dye is a fluorescent dye.

In embodiments, the 3D cell culture medium further comprises one or more extracellular matrix components.

### Example 6

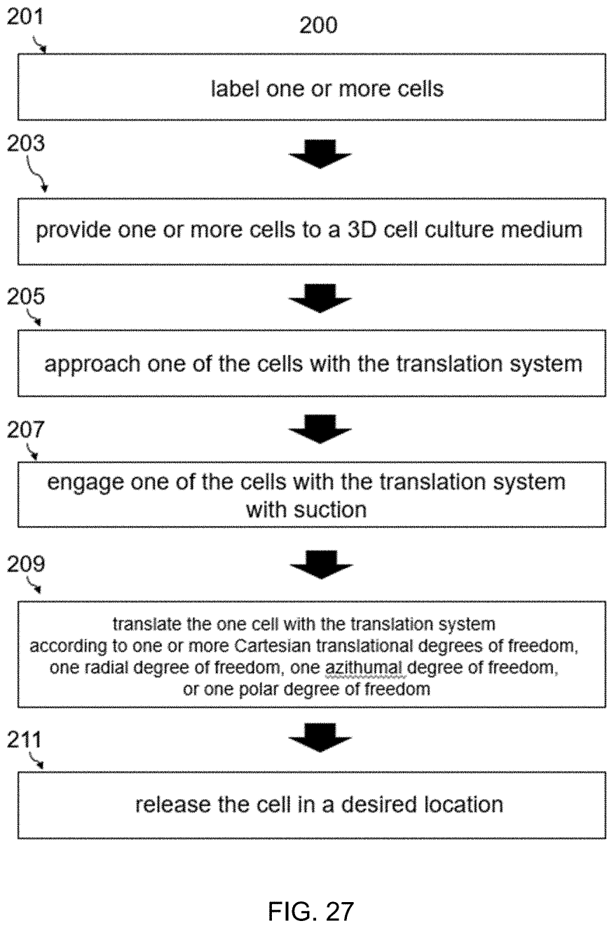

FIG. 27 is a flow chart of an embodiment of a method 200 according to the present disclosure.

In embodiments as described herein, a method 200 of cellular micro-masonry, comprises: labeling one or more cells 201 (with a chemical label or dye, for example); providing one or more cells in the three-dimensional (3D) culture media 203: approaching one of the one or more cells with the translation system 205; engaging the one cell with the translation system using suction 207; translating the one cell with the translation system according to one or more Cartesian translational degrees of freedom, one radial degree of freedom, one azithumal degree of freedom, or one polar degree of freedom 209 (individually or in combination); and releasing the cell in a desired location 211.

### Example 7

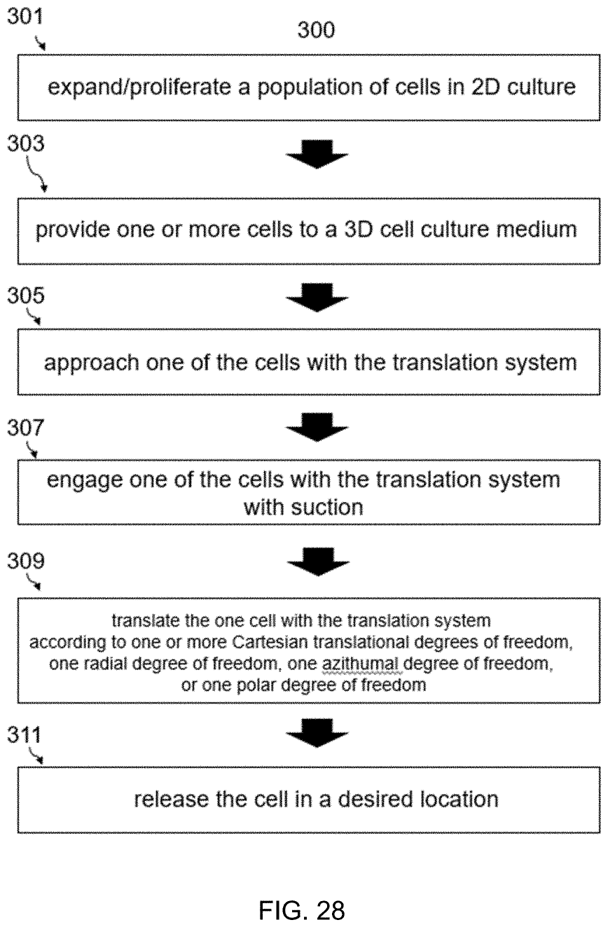

FIG. 28 is a flow chart of an embodiment of a method 300 according to the present disclosure.

In embodiments as described herein, a method 300 of cellular micro-masonry, comprises: expand/proliferate a population of cells in 2D culture 301; providing one or more cells in the three-dimensional (3D) culture media 303: approaching one of the one or more cells with the translation system 305; engaging the one cell with the translation system using suction 307; translating the one cell with the translation system according to one or more Cartesian translational degrees of freedom, one radial degree of freedom, one azithumal degree of freedom, or one polar degree of freedom 309 (individually or in combination); and releasing the cell in a desired location 311. In further aspects, the one or more cells can be labeled with a chemical label/dye before being introduced to the 3D cell culture medium (for example CMFDA or CellMask™ red-orange).

### Example 8

### Abstract:

In many tissues, cell type varies over single-cell length-scales, creating detailed spatial heterogeneities fundamental to physiological function. To gain understanding of this relationship between tissue function and detailed structure, and to one day engineer structurally and physiologically accurate tissues, the ability to assemble 3D cellular structures having the level of detail found in living tissue is needed. Here a method of 3D cell assembly is introduced that has a level of precision finer than the single-cell scale. With this method numerous structures having well-defined spatial patterns can be created, demonstrating that cell type can be varied over the scale of individual cells and showing function after their assembly. This technique provides innumerable opportunities to study cellular behavior in defined contexts including complex interactions operating during embryogenesis.

### Introduction:

The different cell types that constitute living tissue are often structured into highly heterogeneous and complex spatial patterns; cell type can differ over length-scales as small as a single cell within a given tissue (1, 2). This spatial heterogeneity is broadly linked to different types of tissue function. For example, to maintain high rates of molecular exchange in the liver, a network of endothelial cells, called the sinusoid, permeates the periportal zone where every hepatocyte can be found within one or two cell diameters of an endothelial capillary (3, 4). Another dramatic example is found in the pancreatic islet, where the five main cell types of the islet are located within a few cell diameters of one another, making frequent contacts with acinar and ductal cells of the exocrine pancreas (5, 6). This highly heterogeneous arrangement of cell types within the islet's relatively small volume allows cells to communicate through secretion and maintain blood glucose homeostasis (5, 6). The connection between small-scale structural heterogeneity and tissue function is also exhibited by glandular acini in vitro (7-9). These hollow spheres are made from single epithelial monolayers surrounded by a basement membrane; the signaling between the basement membrane and cell nuclei is crucial for acini to develop and function (7, 10-12). While glandular acini represent an in vitro system in which the link between tissue structure and function can be studied in detail, it remains exceedingly challenging to reproduce the complex cellular patterns found more generally in vivo. For example, relying on spontaneous or guided processes of multi-cellular self-assembly within bioengineered tissues is time consuming and does not precisely reproduce the detailed structural and functional heterogeneity at the single-cell scale found within in vivo tissues (13, 14). 3D bioprinting provides control and repeatability for structuring in vitro tissue models, but current tools are not sufficiently precise to produce spatial variations in cell type over the scale of even a few cells, much less a single cell. To create tissue models that reproduce the spatial heterogeneities found within in vivo tissue, new biofabrication tools with single-cell precision are needed. Without such tools, the collective basic understanding of how tissue function collectively emerges from spatially heterogeneous tissue structure will continue to depend on observations and methods that rely dominantly on cell-directed organization, which have persistently challenged researchers.

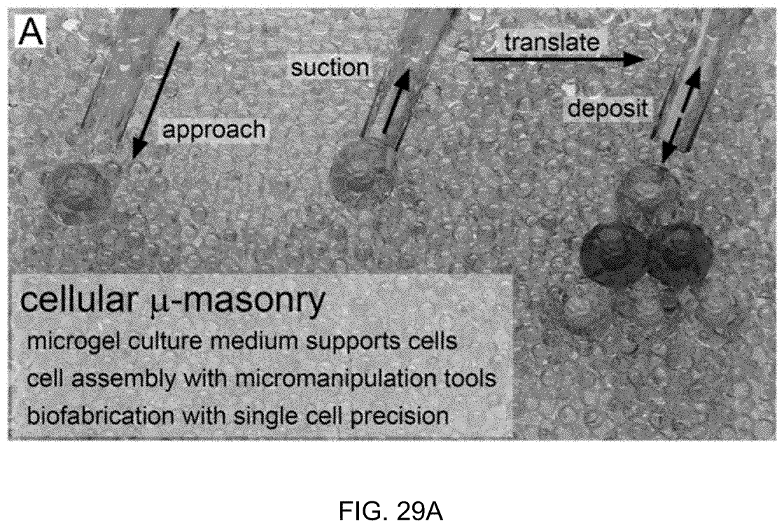

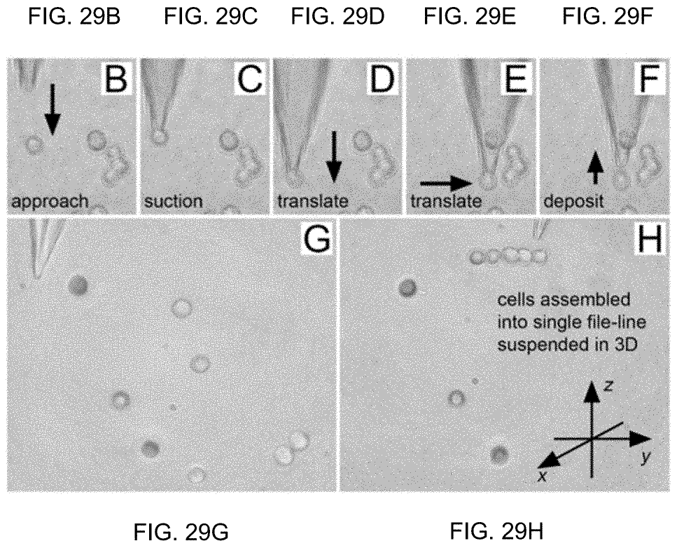

Here a method is introduced that enables one to emulate the heterogeneous spatial patterning found in vivo with single-cell precision. In an embodiment, the method, which can be called “cellular micromasonry,” can combine a soft 3D support medium with micromanipulation and 3D microscopy. The 3D support medium is a phase of soft matter made from jammed granular-scale microgels—hydrogel microparticles packed together that form the microscopic equivalent of the “ball pit” children play in (15-17). Children in ball pits can lay still, supported by the static forces of the packed balls, yet they can also swim through the balls, embedding themselves deep within their surroundings. By analogy, micromanipulators can be used to grasp, translate, and place cells in “ball pit” made from microscopic hydrogel particles swollen in liquid cell growth media (FIG. 29A). This microgel medium is stiff enough to support the cellular structures, but soft enough that a microcapillary holding cells can easily be translated through it; the microgels' low polymer concentration limits the physical stress that cells experience building progresses with them (15-18). In certain aspects, this method can be used to methodically place cells in patterns that comprise heterogeneous populations, controllably alternating between different cells, one-by-one. With this approach, the highest degree of spatial heterogeneity can be achieved—single cell precision. To test for function, molecular transport was studied through gap junctions, observing calcein dye diffusing from cell-to-cell, and it is shown that glandular acini can develop within this medium. This cellular micromasonry method enables the building of stratified, precise cellular structures for detailed investigations of the relationship between structure and function in models of both developing and mature tissues.

### Results

To create precise cellular structures, cells can be harvested from their traditional culture conditions and manually disperse them with a pipette into the microgel culture medium contained in a glass-bottomed petri dish (see Methods for cell types and culture details). The dish is mounted onto a temperature-controlled stage atop an inverted laser scanning confocal microscope. When using carbonate-based culture buffers, humidified CO₂ is gently blown onto the sample surface to maintain neutral pH in the microgel culture medium. Using the microscope, a chosen cell can be identified, translated the tip of a microcapillary to its surface using the micromanipulator, and lightly aspirated using a CellTram® (Eppendorf), applying suction. Once the cell is captured, it can be translated to the desired location and deposited. By repeatedly capturing, translating, and depositing cells, structures suspended in 3D space can be assembled without having to build up from a solid support (FIGS. 29B-29F).

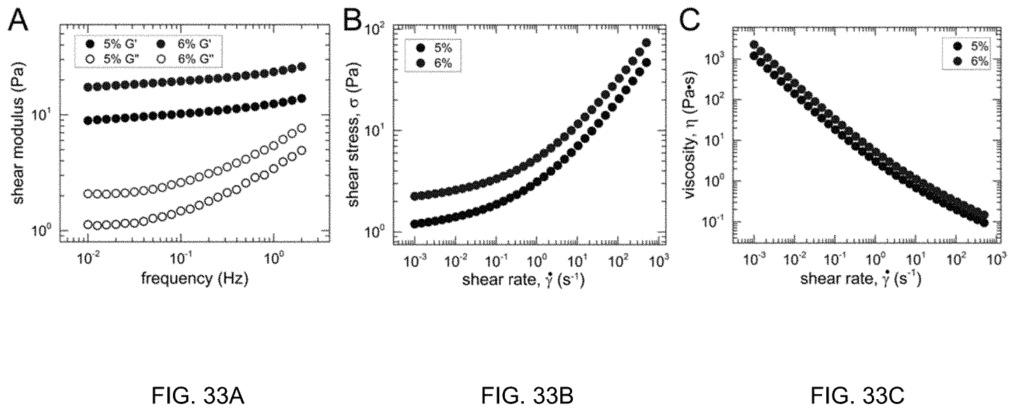

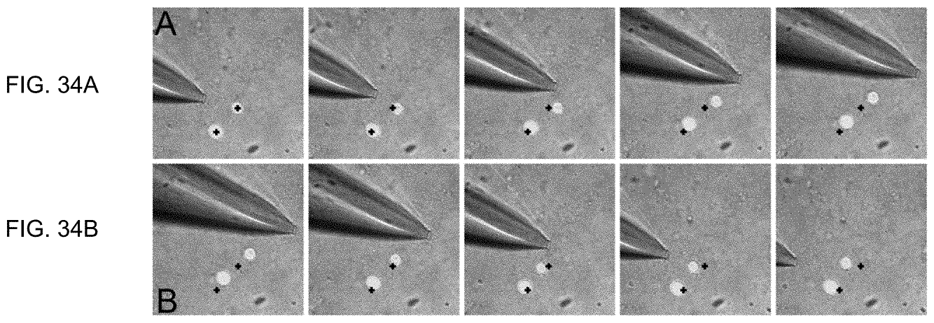

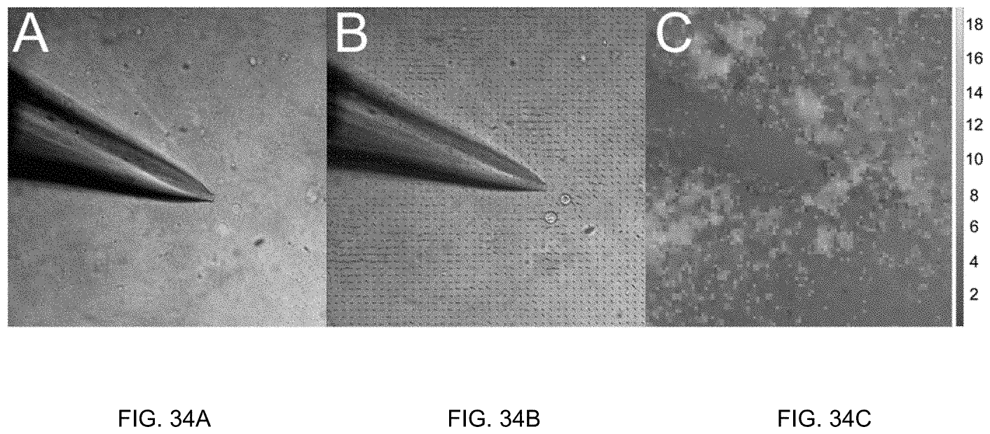

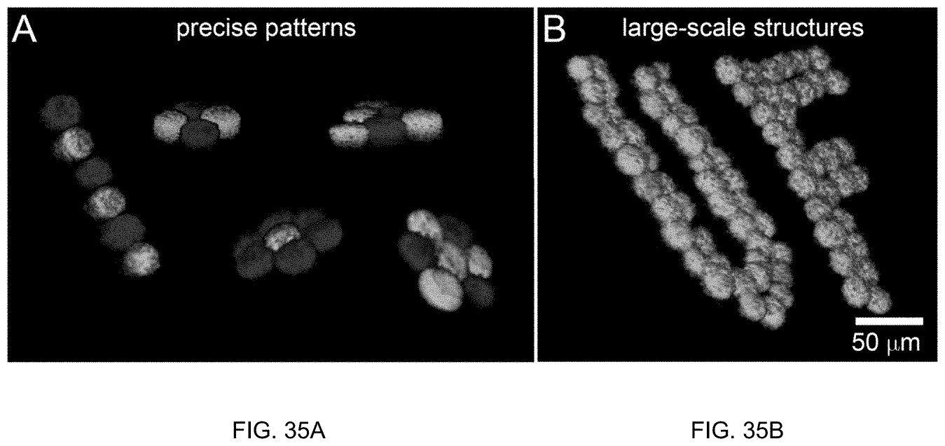

To provide support to cells while minimizing the physical shear stress they endure as structures are built, ensuring the cells are gently cradled in their 3D microenvironment, the microgel medium can be optimized through rheological testing. In embodiments, microgel media formulated at 5-6 wt % polymer has an elastic shear modulus of 10-20 Pa and a yield stress of 1.3-2.4 Pa (see FIGS. 33A-33C and Methods for microgel polymer species). This level of shear stress is comparable to experimental fluid stresses typically imposed on cell surfaces, so it is not expected that this procedure to lead to cell damage (19). As the needle translates back and forth throughout the micromasonry process of building structures, the microgel “balls” are forced to rearrange and flow around the needle surface. To determine whether these rearrangements lead to irreversible, long ranged, or unpredictable flow patterns in the microgel medium, video imaging of a microcapillary translating through the microgel medium containing dispersed cells can be performed, moving at approximately 0.5 mm/s, which is the rate the microcapillary can be translated during micromasonry procedures. It can be found that when the microcapillary is reciprocally translated near suspended cells, the net cell displacement is approximately one cell diameter or less; cells further from the capillary exhibit less hysteresis than cells directly in the path of the microcapillary (FIGS. 34A-34B).

Quantitative analysis of the microgel flow-field around the translating microcapillary helps to explain this reversibility in microgel displacement during the micromasonry process (FIGS. 29A-29F). To understand this apparent hydrodynamic reversibility of the micromasonry method, the Reynolds' number can be estimated, Re, given by ρvd/n, where ρ0 is the microgel mass density, v and d are the microcapillary translation speed and diameter, respectively, and n is the medium viscosity (20). The microcapillary diameter, d, is 1 mm along its shaft and approximately 5 μm near its tip, so the shear-rate range can be approximated to be v/d≈0.5−100 s⁻¹. The corresponding microgel viscosity range from rheological measurements is 0.2-10 Pa s (FIG. 33C). Thus, the maximum Re occurring during micromasonry can be estimated to be approximately 10⁻³, four orders of magnitude below the flow regime where hydrodynamic reversibility begins to break down (21). Consequently, the predictable flow behavior of the packed microgel medium enables the assembly of precise structures in 3D space, like single-file lines of cells (FIGS. 29G,H).

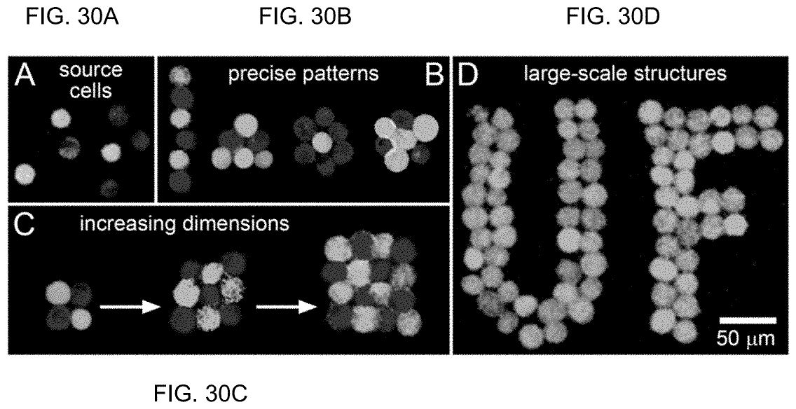

The heterogeneous composition of tissues in vivo often exhibit cell-type variations over length scales as small as the individual cell; to mimic this extreme variability in vitro, structures can be built from cells labeled with different fluorescent dyes. Two populations of Madin-Darby Canine Kidney (MDCK) epithelial cells are cultured under standard 2D conditions, labeling one population with CellMask orange and the other with 5-chloromethylfluorescein diacetate (CMFDA). To create a source population of cells to build with, the cells are harvested and suspended in a glass-bottom petri dish filled with 2 mL of the microgel culture medium. The dish is placed on an inverted confocal microscope equipped with an incubating plate, keeping the cells at 37 C (FIG. 30A). To test the generation of a diversity of different spatial patterns that may occur in different tissues, several basic structures can be assembled: a line of alternating colors, a triangle with rows of alternating color, a six-fold packing of red cells around a central green cell, and the unit cell of a honeycomb lattice (FIG. 30B). Micromasonry can be used, for example, to study the emergence of collective behavior as a function of tissue size. To demonstrate this capability, different sized structures of the same repeating checkerboard pattern were built (FIG. 30C). While some highly ordered tissues exhibit regular patterns like those shown here, even these highly ordered structures often lay on curved manifolds in space (22). For example, the organ of Corti exhibits highly ordered checkerboard patterning and hexagonal packing over the curved surface of the cochlear duct (22). Thus, to explore the possibility of using micromasonry to build larger objects with larger-scale structural complexity than simple geometric shapes, the initials of the Applicant, ‘UF’ were constructed out of almost 100 cells, suspended in 3D space (FIG. 30D). Taken together, these tests of patterning at the single-cell scale and complex structuring at the large-scale demonstrate the potential for using micromasonry to mimic the complexity of in vivo tissues.

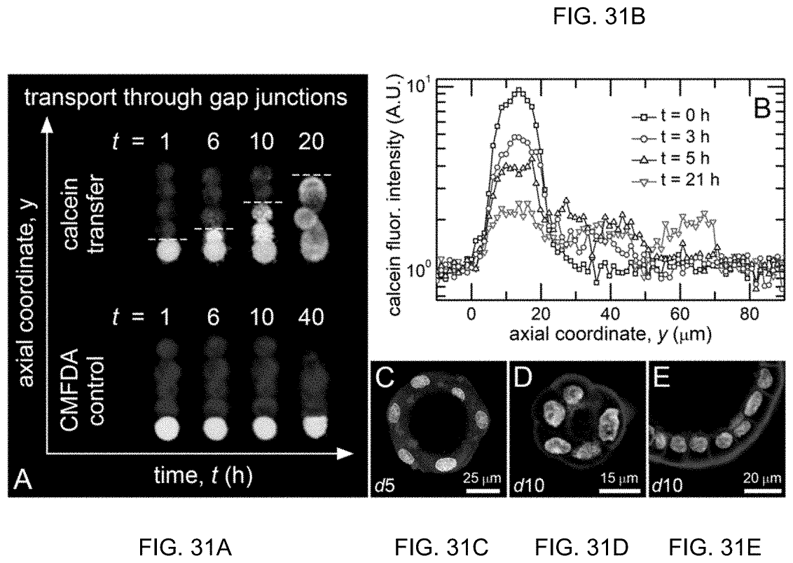

To determine if the cells in these fabricated structures are functionally interacting with one another, a calcein dye assay can be used to test whether gap junctions form. Gap junctions are plaques of intercellular nano-channels that form between neighboring cells that allow the diffusion of small molecules from cell to cell. This transport can be visualized using calcein acetoxymethyl (AM) ester (calcein AM), a cell-permeant live cell dye (23). In live cells, calcein AM is converted to freely diffusing green fluorescent calcein through acetoxymethyl ester hydrolysis, intracellularly. When gap junctions are present, calcein dye can be observed passing from cell to cell with fluorescence microscopy (23). MDCK cells can be cultured in 2D, separate populations can be dyed, for example, with CellMask™ orange and calcein AM, harvest the cells, and randomly disperse both populations as described above. Using the micromasonry technique, single-file lines of four red cells can be built and then a single green cell can be added to the end of the line (FIG. 31A). A confocal Z-stack is taken every 30 minutes for 24 hours. Throughout this period of time the green dye can be seen to travel down the line of cells, indicating that gap junctions indeed form in these manufactured cellular structures (FIG. 31B). It is found, in an example, that the calcein dye takes about 5 hours to travel from cell to cell, which agrees with results from standard calcein assays (23). To ensure this observation requires gap junction permeable fluorophores, control experiments can be performed in which CMFDA is used in place of calcein; CMFDA cannot pass through gap junctions. In these control experiments, the green dye does not spread from cell to cell (FIG. 31C). These results demonstrate that structures suspended in the 3D microgel microenvironment, assembled with the micromasonry technique, maintain their capacity to form the functional gap junctions typically observed in more traditional culture contexts.

As a second functional assay, it can be tested whether glandular acini can form within microgel media. Acini represent one of the best established and widely used tissue models; acini formed from mammary epithelial cells are used in breast cancer research, for example (7-10). Paralleling standard protocols, MDCK cells can be dispersed into a modified microgel media that is swollen in diluted Matrigel (see Methods). After about 10 days of incubation, single cells can be seen proliferating and self-assembling into the monolayer shell structures characteristic of traditionally cultured acini. To compare the architecture of these epithelial shells to traditional acini, cells/structures can be fixed and stained with Hoeschst 33342 and Alexa Fluor® 594 phalloidin to visualize the nucleus and actin cytoskeleton. The stained tissues are then imaged with confocal microscopy where the characteristic monolayer shell structure can be seen as well as the polarized cytoskeletal structure typically found in acini (FIGS. 31C-31E). Slices through the 3D confocal stacks exhibit actin assembly near the outer-facing surface of the shell where a basement membrane is known to form (7-10). These results represent a new way to culture glandular acini and point toward a future path of rich exploration; the cells' mechanical microenvironment can be tuned by preparing the microgel medium at different concentrations, and the granular nature of the microgel medium allows for the micromasonry technique to be combined with spontaneous acini formation. For example, different cell types can be delivered to the maturing acini at chosen locations and times, or concentrated doses of growth factors or other stimulatory molecules can be locally perfused with the micromasonry instrument. Similarly, such a hybrid approach could be used to expand the experimental toolbox for broader investigation of tissue models of healthy development or disease processes (24, 25).

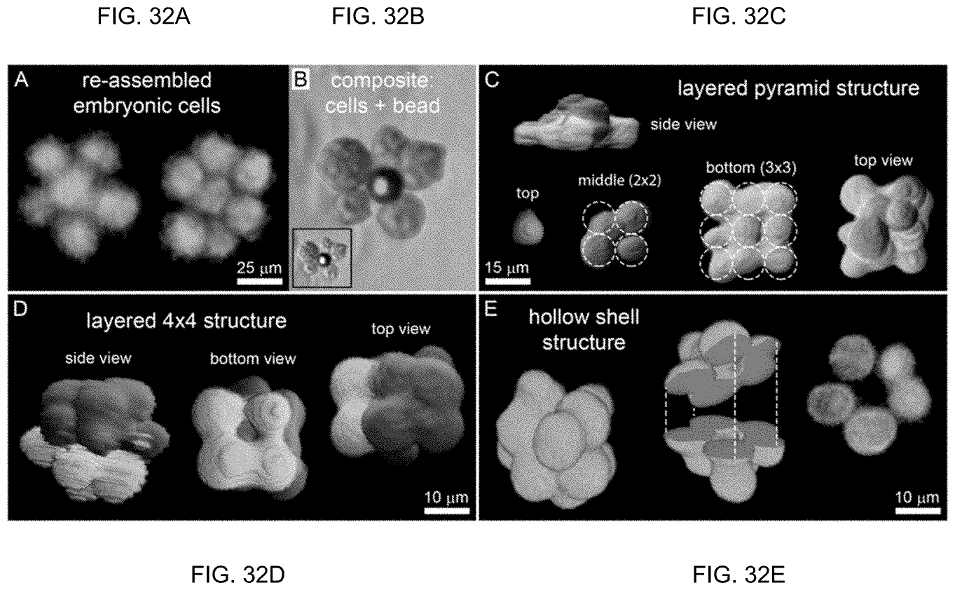

To take the first steps toward using micromasonry for studying more complex cellular structures and potentially manipulating their function, pluripotent embryonic cells can be packed around a functionalized microsphere. Following established protocols, blastula stage embryos (24 hours post fertilization) from the starlet sea anemone, Nematostella vectensis, injected with mRNA for green fluorescent protein as zygotes, are dissociated into single cells in calcium and magnesium free sea water. The dissociated cells are manually collected with a micropipette and dispersed in the microgel medium. Since these embryos are cultured in sea water, a zwitterionic microgel formulation can be developed that does not de-swell at high salt concentrations (see Methods). Several simple planar structures were first constructed from the dissociated cells (FIG. 32A). Fibronectin-coated polystyrene microspheres were then dispersed in the microgel medium and then a hybrid biotic/abiotic structure was built in which the dissociated embryonic cells were deposited on the bead's surface (FIG. 32B). Time-lapse microscopy revealed the cells remained viable and motile, actively spreading on the bead over the course of 13 hours. While further investigation is needed to study how these cells respond to a process of disassembly and re-assembly on a foreign surface, this first demonstration of hybrid biotic/abiotic assembly is key to developing advanced biomaterials that precisely combine living cells with engineered microstructures (26).

To test for the potential of using micromasonry to build layered 3D structures having heterogeneities over length-scales of single cells, approximating the level of detail found in dense living tissues, a series of stratified objects can be built from three separate cell populations. Extending the methods described above, MDCK cells can be labeled with blue, green, and red dyes and then chosen cells can be selectively retrieved from a randomly dispersed population and build layered patterns. A square pyramid structure can be built by assembling a planar 3×3 square packing of green cells, followed by a 2×2 layer of red cells, finishing with a single blue cell at the apex (FIG. 32C). Similarly, a stack of 2×2 layers can be built, with green cells forming the base, blue cells forming the middle layer, and red cells on top (FIG. 32D). Although there are slight imperfections in both structures, no imperfection is more than a single cell diameter, indicating that extremely precise, multicellular structures can be built using this method. As a final test of the potential for using micromasonry to build tissue models, a spherical shell of MDCK cells can be assembled to approximate a glandular acinus. 3D renderings of this assembly reveal its shell structure; slices through the 3D structure show the open pore-space inside the shell of cells (FIG. 32E). While these structures were imaged immediately after building, functional assays of MDCK structures showing gap junction formation and the development of characteristic acini structure over time indicate that these more complex 3D structures may evolve into functional tissue models (FIGS. 31A-31E). It is envisioned that the combination of micromasonry and optimal culture conditions can facilitate building “acini on demand” where the rapid integration of structural and microenvironmental cues accelerate the development of mature acini, with the possibility of extending this principle to other tissue models.

### CONCLUSION

In the present embodiment, all the cellular structures shown here were assembled by operating the micromasonry system by hand, in which a researcher dispersed cells into the microgel medium with a pipette, identified individual cells by eye on a microscope, and meticulously assembled them into the targeted designs by turning dials on micromanipulator control hardware. While single-cell precision was achieved with this manual approach, the current procedure limits the physical scale of structures that can be assembled. However, II the steps in cellular micromasonry can be automated by combining a diversity of current engineering tools like 3D image segmentation, 3D cell tracking, and the control algorithms of pick-and-place robotics (27). Other computational tools based on machine learning and artificial intelligence for controlling machine operations in uncertain environments may also be employed as a path forward to rapidly build larger and more complex structures with an even higher level of precision than that demonstrated here (27).

As improved micromasonry tools are developed for creating larger-scale structures, smaller-scale manual micromasonry can now be used to explore the structure-function relationship in model tissues. Cell to cell signaling occurs extensively throughout embryonic development, with responding cells changing their cell fate and behavior in response to embryonic “organizing centers”. In some embryos these organizing cells can be reduced to a single identifiable cell that establishes the fate of all surrounding cells (28. Lanza and Seaver.). By dissociating developing embryos and using the micromasonry technique to deliver the organizing cell to different complements and spatial distributions of surrounding “responding” cells, the sensitivity of cell signaling to cell positioning in these processes can be quantitatively investigated for the first time. Furthermore, the ability to easily manipulate gene expression (e.g. ligands, receptors, extracellular matrix associated molecules, transcription factors) in model developmental systems such as Nematostella provide powerful opportunities to understand the biology and biophysics of cell-cell interactions (29, Layden et al.) Similarly, a hybrid approach to organoid engineering could be developed. A dominating paradigm in organoid research is to program successive stages of differentiation into pluripotent cells that will spontaneously and collectively mature into functioning differentiated cellular structures that approximate mature organ behavior (30). By building pre-structured assemblies from pluripotent cells and delivering additional programmed cells at precisely chosen locations at critical time-points, the organoid maturation process could be rapidly and controllably guided down many steps of differentiation and development. Finally, with the ability to create cellular structures having crystalline symmetry and spacing, as shown in FIGS. 30A-30D, the tools of synthetic biology could be used to create collective phases of cellular behavior that cannot be achieved within randomly structured cell assemblies. For example, cells can be programmed to secrete molecules at a periodic rate, introducing the possibility of coupling temporal oscillations of signaling molecule concentration with the spatial frequency of cell location. These “cell crystals” could be assembled with the microsmasonry method to synchronize their behaviors with a level of coherence and function that would be destroyed by random cell patterning.

### Materials and Methods

### Jammed Microdel Synthesis

To synthesize polyacrylamide microgels with 17 mol % methacrylic acid, a solution of 8% (w/w) acrylamide, 2% (w/w) methacrylic acid, 1% (w/w) poly(ethylene glycol) diacrylate (MW=700 g mol−1), and 0.1% (w/w) azobisisobutyronitrile in ethanol (490 mL) is prepared [1, 2]. The prepared solution is sparged with nitrogen for 30 min, then placed into a preheated oil bath set at 60° C. After approximately 30 min, the solution becomes hazy and a white precipitate begins to form. The reaction mixture is heated for an additional 4 hours. At this time, the precipitate is collected by vacuum filtration and rinsed with ethanol on the filter. The microparticles are triturated with 500 mL of ethanol overnight. The solids are again collected by vacuum filtration and dried on the filter for ˜10 min. The particles are dried completely in a vacuum oven set at 50° C. to yield a loose white powder. The purified microgel powder is dispersed in cell growth media at various concentrations and mixed at 3500 rpm in a centrifugal speed mixer in 5-min intervals until no aggregates are apparent (17, 18). The microgel is then neutralized to a pH of 7.4 with NaOH and 25 mM HEPES buffer (Part no. BP299-100) and is left to swell overnight, yielding microgel growth media at concentrations of 5-6% (w/w).

In the case of embryonic experiments, zwitterionic microgels are synthesized in the same method from a solution of acrylamide (AAm), poly(ethylene glycol) diacrylate (PEGda) (MW=700 g mol⁻¹), azobisisobutyronitrile (AIBN), and carboxybetaine methacrylate (CBMA) as an ionisable comonomer in ethanol (490 mL) (15, 16). The purified microgel powder is dispersed in sea water at 8% polymer concentration and mixed at 3500 rpm in a centrifugal speed mixer in five-minute intervals until no aggregates are apparent (17, 18). The microgel is then left to swell overnight, yielding microgel the 3D growth media.

### Cell Culture

MDCK cells (Madin Darby Canine Kidney epithelial cells, NBL-2 ATCC CCL-34) are cultured in Dulbecco's Modified Eagle Medium (DMEM) with 4.5 g/L glucose, L-glutamine, and sodium pyruvate supplemented with 10% FBS and 1% penicillin streptomycin in a 12 well plate. When the cells have reached 70% confluence, a single well was dyed with live cell dye. For control studies one well was dyed with CellMask orange plasma membrane stain (Thermo-Fisher, part no. C10045) and a separate well with CellTracker green (CMFDA) (Thermo-Fisher, part no. C2925). For gap junction investigations, one well was dyed with CellMask orange and a separate well with CellTraceCalcein Green AM (Thermo-Fisher, part no. C34852), which becomes fluorescent green in live cells. The wells were passaged separately by washing with PBS, and incubated in 3 mL of 5% Trypsin—EDTA solution for 5 minutes. The cells are harvested from the plate and placed into separate 15 mL centrifuge tubes, where they are centrifuged at 650g for 4 minutes. The supernatant is removed and each pellet is suspended in 1 mL of fresh cell growth media. The cell pellets are dispersed with gentle pipette mixing and 100 μL of each solution is placed in a fresh 15 mL tube and mixed together with light pipetting. About 50 μL of the solution is dispersed in the jammed microgel using a pipette. The microgel 3D culture medium is prepared for each cell type using the corresponding liquid media. 35-mm glass bottom petri dishes are used to facilitate fluorescence imaging. 3 mL of microgel media is loaded into each well. Prior to adding cells, dishes containing microgel media are incubated at 37° C. and 5% CO₂.

### Building Single Cell Structures

Once the cell loaded petri dish is prepared, it is moved to the confocal microscope. An incubating plate is used to keep the dish warm during the building process. A thin layer of oil is placed over the gel to diminish evaporation effects. The needle is slowly lowered into the gel and centered at the correct x, y, and z position using the 4× objective. A ‘layout’ image is taken using the fluorescent microscope at 10× magnification; this allows the user to visualize cell locations and colors for specific builds. Using the 10× objective and 1.5 zoom, structures are built referencing the ‘layout’ cell-by-cell by applying light pressure, moving the needle and releasing pressure to place the cells in 3D space. Over about 24 hours, a fluorescent z stack is taken every 30 minutes.

Embryo Experiments

Nemaotstella vectensis embryos were obtained from a breeding colony in the Martindale lab at the Whitney Lab for Marine Bioscience (Univ. Florida). Zygotes were deljellied and injected with mRNAs to GFP or mCherry to generate fluorescently labeled blastomeres. Embryos were grown to blastula stages (24-48 hours post fertilization) and dissociated into individual cells using calcium/magnesium free seawater plus 5 mM egtazic acid (EGTA). The embryos are placed in a petri dish and ˜2 mL of the dissociation solution is added, the embryos are mechanical dissociated using a pipette for 1 minute or until there are no large pieces of tissue visible. The dissociated embryo solution is transferred to a 40 μm cell strainer positioned over a clean petri dish and washed with the dilution solution.

The dissociated embryos were manually injected into the zwitterionic microgels, synthesized as described above, swollen with the dilution solution and transferred to the microscope stage. A thin layer of mineral oil is added on top of the microgel to decrease evaporation and the petri dish is brought to the confocal microscope. The needle tip, pulled to about 10 μm, is slowly lowered into the gel and carefully centered using the 4× objective then lowered to the ideal Z height. A ‘layout’ image is taken to determine the location of embryonic cells and each cell is picked up with light suction, transferred and placed to create a circular structure, the structure is observed to determine how the cells reorganized over time. In some of the experiments, polystyrene beads are coated in 1 mg/mL fibronectin, dispersed in the microgel, and cells are place around the bead.

Rheology of Jammed Microgels

The methacrylic acid microgel particles are synthesized in house, as described in Materials and Methods, and swollen in DMEM supplemented with 5% FBS and 1% penicillin streptomycin, providing the necessary nutrients for cells to function. To determine the material and flow properties of the jammed microgels, traditional rheological tests can be conducted including frequency sweeps (1% strain amplitude) and unidirectional shear-rate sweeps (FIGS. 33A-33C). It can be found that microgel media prepared within a polymer concentration range of 5-6% is well suited for use with the micromasonry technique. Within this range, the microgel media has an elastic shear modulus of 10 to 20 Pa and a yield stress of 1.3-2.4 Pa. The stresses measured in shear-rate sweeps are often divided by the shear-rate to determine the viscosity. While the microgel media is dominantly solid-like at low shear-rates, at high shear rates the microgel media becomes a shear-thinning fluid, exhibiting a decreasing viscosity with increasing shear-rate. This viscosity curve can be used to estimate the range of Reynold's number the media exhibits during micromasonry procedures.

### Microgel Flow and Reversible Cell Displacement Near Translating Capillary Tips

During micromasonry procedures, it is possible that the microgel medium flows excessively and that dispersed cells displace irreversibly because of material's rheological properties. To investigate the degree of microgel flow and irreversible cell displacement during micromasonry procedures, a series of tests can be performed in which the microcapillary is translated back and forth near suspended cells. Bright-field and fluorescence videos are collected throughout the tests to monitor the motion of the microcapillary, the cells, and the supporting microgel medium (FIGS. 34A-34B). After one cycle, it can be seen that the cells displace by less than one cell diameter. Since the microgel medium is inherently an optically grainy material, particle image velocimetry (PIV) can be used to measure the entire displacement field after one cycle (FIGS. 34A-34B). In an embodiment, the root-mean-square (RMS) displacement, averaged over the entire field of view, to be 12 μm, which is less than a single cell diameter.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A cellular micro-masonry system, comprising:
a translation system;
an imaging system; and
a three-dimensional (3D) culture medium wherein the 3D cell culture medium comprises a plurality of hydrogel particles and a liquid cell culture medium, wherein the hydrogel particles are swelled with the liquid cell culture medium to form a granular gel.

2. The cellular micro-masonry system of claim 1, further comprising a suction generating system, a pressure generating system, or both coupled to the translation system.

3. The cellular micro-masonry system of claim 1, wherein the translation system further comprises a micro-capillary.

4. The cellular micro-masonry system of claim 1, wherein the translation system is configured to provide one or more of three cartesian translational degrees of freedom (X, Y, Z), one radial degree of freedom (R), one azimuthal degree of freedom (ϕ), and one polar degree of freedom (θ).

5. The system of claim 1, wherein the 3D culture medium has a yield stress such that the cell growth medium undergoes a phase change from a first solid phase to a second liquid phase upon application of a shear stress greater than the yield stress.

6. The system of claim 1, wherein the concentration of hydrogel particles is between 0.05% to about 1.0% by weight.

7. The system of claim 1, wherein the hydrogel particles have a size between about 0.1 μm to about 100 μm when swollen with the liquid cell culture medium.

8. The system claim 1, wherein the 3D cell culture medium further comprises one or more extracellular matrix components.

9. The system of claim 1, wherein the hydrogel particles are comprised of zwitterionic microgels.

10. A method of cellular micro-masonry, comprising:
providing a cellular micro-masonry system;
providing one or more cells in the three-dimensional (3D) culture media:
approaching one of the one or more cells with the translation system;
engaging the one cell with the translation system using suction;
translating the one cell with the translation system according to one or more Cartesian translational degrees of freedom, one radial degree of freedom, one azithumal degree of freedom, or one polar degree of freedom; and
releasing the cell in a desired location.

11. The method of claim 10, further comprising manually correcting errors before or after the releasing.

12. The method of claim 10, further comprising discarding cells that are not suitable.

13. The method of claim 10, wherein the approaching, engaging, translating, and releasing are monitored by the user using an imaging system.

14. The method of claim 10, wherein the 3D culture medium has a yield stress such that the cell growth medium undergoes a phase change from a first solid phase to a second liquid phase upon application of a shear stress greater than the yield stress.

15. The method of claim 10, wherein the concentration of hydrogel particles is between 0.05% to about 1.0% by weight.

16. The method of claim 10, wherein the one or more cells are one or more tumor cells.

17. The method of claim 10, further comprising proliferating the one or more cells in 2D culture before providing them to the 3D culture medium.

18. The method of claim 10, further comprising labeling the one or more cells with a live-cell dye.

19. The method of claim 10, wherein the 3D cell culture medium further comprises one or more extracellular matrix components.

20. The system of claim 1, wherein the 3D culture media comprises zwitterionic microgels.
